(19) United States
(12) Patent Application Publication
(10) Pub. No.: US 2022/0241205 A1
(43) Pub. Date: Aug. 4, 2022

(54) Improved Lyophilized Formulations Involving Hyaluronic Acid and Plasmatic Proteins, and Uses Thereof

(71) Applicant: BONE THERAPEUTICS SA, Gosselies (BE)

(72) Inventors: Nadia Stricwant, Thuillies (BE); Jean Didelez, Court-Saint-Etienne (BE)

(21) Appl. No.: 17/610,254

(22) PCT Filed: May 13, 2020
(86) PCT No.: PCT/EP2020/063302

§ 371 Date: Nov. 10, 2021

(30) Foreign Application Priority Data
May 13, 2019 (EP) 19174129.7

(51) Int. Cl. A61K 9/19; A61K 31/728; A61K 9/00; C08B 37/08; A61L 27/54

(52) U.S. Cl. CPC A61K 9/19; A61K 31/728; A61L 2300/436; C08B 37/0072; A61L 27/54; A61K 9/0019

(57) ABSTRACT

The invention concerns lyophilized pharmaceutical formulations comprising plasmatic proteins or derivatives thereof, hyaluronic acid or a derivative thereof, and optionally one or more other pharmaceutically active ingredients, such as clonidine or a derivative thereof, wherein the lyophilized pharmaceutical formulations are characterized by a reconstitution time of less than 15 minutes. The invention further relates to methods for preparing these formulations, and their use for treating musculoskeletal diseases such as bone or joint diseases.

### CROSS REFERENCE TO RELATED APPLICATIONS

This application is a national phase entry under 35 U.S.C. § 371 of International Patent Application PCT/EP2020/063302, filed May 13, 2020, designating the United States of America and published in English as International Patent Publication WO 2020/229526 on Nov. 19, 2020, which claims the benefit under Article 8 of the Patent Cooperation Treaty to European Patent Application Serial No. 19174129.7, filed May 13, 2019, the entireties of which are hereby incorporated by reference.

### FIELD

Aspects of the invention are broadly in the medical therapeutic field and more specifically concern pharmaceutical formulations or kits-of-parts and their use for treating diseases such as musculoskeletal diseases, and more particularly bone or joint diseases.

### BACKGROUND

Musculoskeletal diseases are a group of diseases that affect bones, muscles, cartilage, tendons, ligaments, and other connective tissues. These disorders can develop over time or be the result of excessive use of the musculoskeletal system or from trauma. Recently, there has been a focus on developing liquid formulations for local delivery, and in particular intra- or peri-osseous or intra- or peri-articular delivery of pharmaceutical active ingredients to avoid systemic side effects (WO2014/049063). These liquid formulations may comprise solvent/detergent treated plasma and a glycosaminoglycan. After administration, the formulations may display gel consistency, retaining the pharmaceutical active ingredients and releasing them gradually.

It is therefore a challenging aspect to achieve reduced viscosity of the formulation during the injection and recover viscosity in situ. Several formulations are unstable in an aqueous environment, even when exposed for a short duration and thus require packaging, storing and shipping in a powder or lyophilized state to keep the product stable during its shelf life. However, current lyophilized pharmaceutical formulations suitable for local administration are characterized by long reconstitution times, leading to a cumbersome reconstitution process not optimally suited for use in clinical practices. In addition, the slow reconstitution introduces a risk of administering partially reconstituted formulations leading to unsatisfactory concentrations of pharmaceutical active ingredients being present in the affected part of the body.

There thus exists a continuous need for further and/or improved lyophilized pharmaceutical formulations that have faster reconstitution times.

### SUMMARY

As evidenced in the examples which illustrate certain representative embodiments of the invention, the present invention relates to improved lyophilized pharmaceutical formulations that have a short reconstitution time (≤15 min) addressing one or more of the above-mentioned problems in the art. The findings are unexpected, inter alia because upon lyophilisation of the pharmaceutical formulations comprising hyaluronic acid and plasmatic proteins known in the art for treating musculoskeletal diseases, a lyophilized product is obtained that is characterized by long reconstitution times, rendering their use in routine practice troublesome.

The inventors found that the formulations, methods, and kits of the present invention provide one or more advantages compared to the state of the art. The present invention allows to significantly improve reconstitution times of lyophilized formulations, particularly of lyophilized formulations for the treatment of musculoskeletal diseases. Medical staff administering these formulations is no longer restricted in their practice by long reconstitution times and thus the convenience of using the formulations, methods and kits of the present invention is improved for both patients and medical staff. Furthermore, shorter reconstitution times ensure that the optimal active pharmaceutical dosage is supplied to the patient, as there is a risk with formulations having longer reconstitution times that only partially reconstituted formulations are administered to a patient.

A first aspect of the invention provides a lyophilized pharmaceutical formulation comprising plasmatic proteins or derivatives thereof and hyaluronic acid or a derivative thereof, wherein the formulation, when reconstituted in an aqueous solution, has a reconstitution time of 15 minutes or less.

Preferably, the invention provides a lyophilized pharmaceutical formulation comprising lyophilized plasma and hyaluronic acid or a derivative thereof, wherein the formulation, when reconstituted in an aqueous solution, has a reconstitution time of 15 minutes or less and is configured for injection.

A further aspect of the invention provides a kit-of-parts comprising (a) a lyophilized pharmaceutical formulation as described herein; (b) a syringe comprising an aqueous solution; and (c) preferably, at least one needle. In certain embodiments, the syringe may be a double syringe comprising the lyophilized pharmaceutical formulation as described herein in one compartment and an aqueous solution in a second compartment.

A further aspect of the present invention provides a process for preparing a lyophilized pharmaceutical formulation as described herein, comprising the following steps:
(a) mixing plasmatic proteins or derivatives thereof, hyaluronic acid or derivative thereof, and an aqueous solution, thereby obtaining a bulk mixture having a concentration of the plasmatic proteins or derivatives thereof of 20 mg/ml to 50 mg/ml and a concentration of the hyaluronic acid or derivative thereof of 4 mg/ml to 8 mg/ml;
(b) sterilizing the bulk mixture by steam sterilization or filter sterilization, thereby obtaining a sterile mixture; and
(c) lyophilizing the sterile mixture, thereby obtaining the lyophilized pharmaceutical formulation.

Preferably, the invention provides a process for preparing a lyophilized pharmaceutical formulation as described herein, comprising the following steps:
(a) mixing plasma and hyaluronic acid or derivative thereof, thereby obtaining a bulk mixture;
(b) sterilizing the bulk mixture by steam sterilization or filter sterilization, thereby obtaining a sterile mixture; and
(c) lyophilizing the sterile mixture, thereby obtaining the lyophilized pharmaceutical formulation;

wherein step (a) comprises the steps of (a1) dissolving the hyaluronic acid or derivative in an aqueous solution, thereby obtaining a first solution; (a2) preparing a second solution comprising the plasma, and, optionally, an alpha-2 adrenergic receptor agonist, and (a3) mixing the first and second solution to obtain the bulk mixture.

A further aspect of the invention relates to a lyophilized pharmaceutical formulation obtainable or obtained by a process as described herein.

A further aspect of the invention provides for the lyophilized pharmaceutical formulation as described herein for use in the treatment of a musculoskeletal disease, preferably wherein the lyophilized pharmaceutical formulation is mixed with an aqueous solution prior to administration.

The above and further aspects and preferred embodiments of the invention are described in the following sections and in the appended claims. The subject matter of appended claims is hereby specifically incorporated in this specification.

### BRIEF DESCRIPTION OF THE DRAWINGS

FIG. 1. Visual appearance of three vials comprising a lyophilized pharmaceutical formulation according to an embodiment of the invention.

FIG. 2. Graph represents a representative hydration curve illustrating the weight in function of time for 5 lyophilized pharmaceutical formulations according to an embodiment of the present invention prepared by a method comprising mixing the first solution and the second solution at a ratio of 1:1 (v/v) and with medium molecular weight HA.

### DESCRIPTION OF EMBODIMENTS

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

Lyophilisation of earlier developed liquid pharmaceutical formulations comprising plasmatic proteins and hyaluronic acid, particularly for treating musculoskeletal diseases (WO2014049063), are characterized by long reconstitution times, which is inconvenient for routine use in clinical practices. As evidenced in the examples, which illustrate certain representative embodiments of the invention, the inventors have developed improved lyophilized pharmaceutical formulations and methods to obtain said formulations that have a short reconstitution time (≤15 min), thus addressing one or more of the above-mentioned problems in the art.

The terms “lyophilized” or “freeze-dried” can be used interchangeably herein and refer to a condition and/or state of a sample, formulation, or product obtained by means of lyophilisation. Lyophilisation, also known as freeze-drying or cryodesiccation, is a dehydration process which involves freezing the product without destroying the physical structure of the matter. Lyophilisation comprises at least a freezing step and a sublimation step. The sublimation step may comprise two stages of drying: a primary drying step and a secondary drying step. Lyophilisation may be used in the manufacturing of pharmaceutical products and intermediates thereof. During freezing, the material is cooled to a temperature wherein the solid, liquid, and gas phases of the material may exist. Active pharmaceutical product ingredients (APIs) may be lyophilized to achieve chemical stability allowing room temperature storage. This is different from a conventional method that evaporates water using heat. Advantages of lyophilisation may be but are not limited to improved aseptic handling, enhanced stability of a dry powder, the removal of water without excessive heating of the product, and enhanced product stability in a dry state. In general, the quality of a rehydrated, lyophilized product is excellent and does not show inferior (therapeutic) characteristics to a non-lyophilized product.

The terms “pharmaceutical formulation”, “pharmaceutical composition”, or “pharmaceutical preparation” may be used interchangeably herein. Likewise, the terms “formulation”, “composition”, or “preparation” may be used interchangeably herein. Throughout this description, absolute quantities as referred to herein correspond to the amounts present in one administration dose, unless explicitly stated otherwise.

Preferably, the lyophilized pharmaceutical formulation is a soluble or dissolvable formulation. The lyophilized pharmaceutical formulation advantageously dissolves when reconstituted in an aqueous solution, e.g. all or substantially all, such as at least 90%, at least 95%, at least 96%, at least 97%, at least 98%, at least 99%, at least 99.5% or 100% of the lyophilized pharmaceutical formulation is dissolved or solubilized when reconstituted.

As used herein, “reconstitution” refers to the process of restoring a dried, lyophilized, dehydrated, or concentrated matter to its original or liquid state by adding a solvent to the lyophilized matter, allowing the lyophilized matter to rehydrate, followed by agitating the mixture of the solvent and lyophilized matter. The reconstituted matter may be or may be part of a product, formulation, sample, raw material, or any biological material but is certainly not limited to matter falling under the common definition of these terms. Reconstitution can be assessed visually with the naked eye. The lyophilized matter is deemed reconstituted when a homogeneous solution is observed. In particular, a solution with a cloudy appearance is considered suitably reconstituted.

Alternatively, reconstitution can be assessed by impedance-based methods. In such methods, minor changes in impedance of the reconstitution medium are detected due to the solid material dissolving during the reconstitution or dissolution process. A dual electrode needle is injected in the diluent and the impedance signal change is continuously monitored in the added diluent. It determines concentration levels, as impedance depends on the number and mobility of ionic carriers that allow electrical current to flow. The electric current (I) conducted by a liquid containing singly charged ions can be expressed as: I=N_{qμ}U/L² with N as the number of ions in the liquid, q is the elementary charge, μ is the average ion mobility, U is the applied potential, and L is the length of the conduction path. The electrical resistance can be expressed as R=L²/N_{qμ}. The electrical resistance represents the real part of the impedance signal (Z), which is also dependent on the reactance (X), which in turn is dependent on the applied AC frequency (f) and the solution capacity (c): Z=\sqrt{R2+X2} with X=1/(πfc). All components of a lyophilisate that dissolve during reconstitution will contribute to impedance signal change. The endpoint of reconstitution was defined at an impedance change of less than 1 ohm for at least 7 seconds.

The term “aqueous solution” refers to any solution comprising water or in which the solvent is water. Additionally, “aqueous solution” is used to describe solutions displaying commonalities to water or watery solutions, not limited to characteristics such as appearance, smell, colour, taste, viscosity, pH, absorbance, or physical state under particular temperatures.

The terms “weight percentage”, “mass percentage”, “percentage (%) by weight”, “weight %” or “wt %” indicate the mass of a substance to the total mass of the formulation (i.e. mass fraction) with a denominator of 100. Unless indicated otherwise, the wt % is provided herein compared to the total weight of the lyophilised pharmaceutical formulation.

The term “buffer component”, “buffer solution”, or “buffer” as used interchangeably herein refers to an aqueous solution comprising a mixture of a weak acid and its conjugate base or vice versa. Buffer solutions are characterized by their means to keeping the pH of a solution nearly constant when limited amounts of strong acids or strong bases are added to the solution. The amount of strong acid or strong base that can be added to the buffer solution before a significant pH change occurs is dependent on the specific buffer solution used and is commonly referred to as the buffer capacity. The pH of a buffer solution can be estimated using the Henderson-Hasselbalch equation, which is known to a person skilled in the art.

As defined herein, the pH of a formulation may be measured using various methods as known to a person skilled in the art. pH indicators may be used that discolour by uptake or release of H+-ions, wherein their resulting colour is indicative for a certain pH value. Alternatively, pH meters may be used that measure the difference in electrical potential between a pH electrode and a reference electrode. The difference in electrical potential relates to the acidity or pH of the solution.

A first aspect of the present invention provides a lyophilized pharmaceutical formulation comprising plasmatic proteins or derivatives thereof and hyaluronic acid or a derivative thereof, wherein the formulation, when reconstituted in an aqueous solution, has a reconstitution time of 15 minutes or less, preferably 10 minutes or less, more preferably 5 minutes or less. In particular embodiments, the lyophilized pharmaceutical formulation comprises lyophilized plasma and hyaluronic acid or a derivative thereof, wherein the formulation, when reconstituted in an aqueous solution, has a reconstitution time of 15 minutes or less, preferably 10 minutes or less, more preferably 5 minutes or less.

Accordingly, an aspect relates to a lyophilized pharmaceutical formulation comprising lyophilized plasma and hyaluronic acid or a derivative thereof, wherein the formulation, when reconstituted in an aqueous solution, has a reconstitution time of 15 minutes or less, preferably 10 minutes or less, more preferably 5 minutes or less.

The terms “lyophilized pharmaceutical formulation”, “lyophilized formulation”, “lyophilized cake”, “cake” and “formulation” are used interchangeably herein, and refer to the lyophilised pharmaceutical formulation as taught herein.

In embodiments, the invention provides a lyophilized pharmaceutical formulation comprising plasmatic proteins or derivatives thereof and hyaluronic acid or a derivative thereof, wherein the formulation comprises from about 30% to about 80% by weight of the plasmatic proteins or derivatives thereof, wherein the formulation, when reconstituted in an aqueous solution, has a reconstitution time of 15 minutes or less and is configured for injection.

“Plasmatic proteins” as defined herein refers to plasma derived proteins, or proteins that may be present and/or detected in blood plasma. Plasmatic proteins are not limited to human plasmatic proteins, unless explicitly stated throughout this document. The plasmatic proteins comprised in the pharmaceutical formulation may comprise any protein or modified protein naturally originating in plasma. As used herein, the term “plasmatic proteins” also include synthetic plasmatic proteins or plasmatic protein derivatives.

The recitations “plasmatic protein derivatives” or “derivatives of plasmatic proteins” as described herein refers to single proteins derived from plasma, such as any single one of the plasmatic proteins as listed herein.

In certain embodiments, the plasmatic proteins or derivatives thereof may be derived from plasma. In certain embodiments, the plasmatic proteins or derivatives thereof may be derived from lyophilized plasma. In certain embodiments, the plasmatic proteins or derivatives thereof may be part of lyophilized plasma. In certain embodiments, the lyophilized plasma comprises plasmatic proteins or derivatives thereof.

The term “plasma” is as conventionally defined. Plasma may be any plasma as conventionally defined such as fresh plasma, fresh frozen plasma, thawed frozen plasma, or cryoprecipitate, cryosupernatants or concentrates from frozen plasma as well as dilution products thereof. The term “plasma” also includes PRP (platelet-enriched plasma) or a plasma substitute. Plasma is usually obtained from a sample of whole blood, provided or contacted with an anticoagulant, (e.g., heparin, citrate, oxalate or EDTA). Subsequently, cellular components of the blood sample are separated from the liquid component (plasma) by an appropriate technique, typically by centrifugation. The term “plasma” therefore refers to a composition which does not form part of a human or animal body. In certain embodiments, the plasma may be derived from warm-blooded animals, such as mammalian animals, such as humans.

The term “platelet-rich plasma (PRP)” refers to plasma that has been enriched with platelets.

Typically, PRP may contain about 1.0×10⁶ platelets/μl, whereas platelet concentration in whole blood may be about 1.5×10⁵ to 3.5×10⁵/μL. Accordingly, plasma as intended herein may contain less than about 1.5×10⁵ to 1.0×10⁶ platelets/μL.

In embodiments, the plasma is not platelet-rich plasma. In embodiments, the plasma is not subjected to further enrichment or fractioning steps before being used in the process as taught herein for preparing a lyophilized pharmaceutical formulation. In embodiments, the plasma may have a composition which is substantially the same as plasma obtained in a conventional manner, e.g. as described above.

In certain embodiments, the lyophilized pharmaceutical formulation comprises lyophilized plasma. In certain embodiments, the lyophilized pharmaceutical formulation comprises lyophilized plasma, which in turn comprises plasmatic proteins or derivatives thereof. In certain embodiments, the lyophilized pharmaceutical formulation comprises lyophilized solvent/detergent-treated (S/D) plasma. In certain embodiments, the lyophilized pharmaceutical formulation comprises plasmatic proteins which are solvent/detergent-treated (S/D) plasma proteins. In certain embodiments, the S/D plasma proteins are derived from warm-blooded animals, such as mammalian animals, such as humans.

In certain embodiments, the plasma may be S/D plasma. In certain embodiments, the plasmatic proteins are solvent/detergent-treated (S/D) plasma proteins, preferably human S/D plasma proteins.

In certain embodiments, the plasmatic proteins or derivatives thereof may be derived from S/D plasma. In certain embodiments, the plasmatic proteins or derivatives thereof may be part of lyophilized S/D plasma. In certain embodiments, the lyophilized S/D plasma comprises plasmatic proteins or derivatives thereof.

The terms “solvent/detergent-treated plasma”, “S/D-treated plasma”, or “S/D plasma” generally refer to decellularised plasma obtainable or obtained by a method comprising the steps of: (a) treating plasma with a solvent and a detergent and (b) filtering the solvent/detergent-treated plasma.

The plasma to be treated in step (a) may be any plasma as conventionally defined such as fresh plasma, fresh frozen plasma, thawed frozen plasma, or cryoprecipitate, cryosupernatants or concentrates from frozen plasma as well as dilution products thereof. Plasma is usually obtained from a sample of whole blood, or from a sample obtained by apheresis.

The solvent used for preparing S/D plasma preferably is a dialkylphosphate or a trialkylphosphate, both having alkyl groups which contain 1 to 10 carbon atoms, especially 2 to 10 carbon atoms. Illustrative examples of solvents may include tri-(n-butyl)phosphate, tri-(t-butyl)phosphate, tri-(n-hexyl)phosphate, tri-(2-ethylhexyl)phosphate, or tri-(n-decyl)phosphate. A preferred solvent is tri-(n-butyl)phosphate. The solvent such as di- or trialkylphosphate for use in the treatment step (a) preferably is employed in an amount ranging from about 0.01 mg/ml to about 100 mg/ml, and preferably from about 0.1 mg/ml to about 10 mg/ml. Stated differently, di- or trialkylphosphates for use in the treatment step (a) preferably are employed in an amount ranging from about 0.001% w/v to about 10% w/v, and preferably from about 0.01% w/v to about 1% w/v.

The detergent used for preparing S/D plasma preferably is a non-toxic detergent. Contemplated non-ionic detergents include those which disperse at the prevailing temperature at least 0.1% by weight of the fat in an aqueous solution containing the same when 1 gram detergent per 100 ml of solution is introduced therein. Illustrative examples of detergents may include polyoxyethylene derivatives of fatty acids, partial esters of sorbitol anhydrides, for example, those products known commercially as “Tween® 80”, “Tween® 20” and “polysorbate 80” and non-ionic oil soluble water detergents such as that sold commercially under the trademark “Triton™ X 100” (oxyethylated alkylphenol). Also contemplated is sodium deoxycholate as well as the “Zwittergents” which are synthetic zwitterionic detergents known as “sulfobetaines” such as N-dodecyl-N,N-methyl-2-ammonio-1 ethane sulphonate and its congeners or non-ionic detergents such as octyl-beta-D-glucopyranoside. The amount of detergent may range from about 0.001% v/v to about 10% v/v, preferably from about 0.01% v/v to 1.5% v/v.

The treatment with solvent and detergent preferably is effected at a temperature between −5° C. and 70° C., preferably between 0° C. and 60° C. The time of such treatment (contact) is at least 1 minute, preferably at least 1 hour and generally 4 to 24 hours. The treatment is normally effective at atmospheric pressure, although sub-atmospheric and super-atmospheric pressures may also be employed.

Normally, after the treatment, the solvent such as trialkylphosphate and the detergent are removed. The solvent and detergent may be removed by any technique suitable for separating the solvent and detergent from the plasma. When a non-ionic detergent is employed with the solvent such as trialkylphosphate, they may be removed by: (1) diafiltration using microporous membranes such as TEFLON which retain the plasma proteins; (2) absorption of desired plasma components on chromatographic or affinity chromatographic supports; (3) precipitation, for example, by salting out of plasma proteins; (4) lyophilisation, etc. Solvents such as dialkylphosphate or trialkylphosphate may be removed as follows: (a) removal from antihemophilic factor (AHF) may be effected by precipitation of AHF with 2.2 molar (M) glycine and 2.0 M sodium chloride (b) removal from fibronectin may be effected by binding the fibronectin on a column of insolubilized gelatine and washing the bound fibronectin free of reagent.

The filtering step (b) is generally performed with a 1 μm filter to remove cells and debris, followed by sterile filtration using a 0.2 μm filter.

In certain embodiments, the S/D treatment comprises at least one solvent and/or detergent extraction step by using oil. Preferably, the oil is soybean oil or castor oil. In certain embodiments, the plasma is further treated by an additional process prior or after S/D treatment. In further embodiments, these processes may comprise ultraviolet (UV)-radiation alone or in combination with a photochemical active agent. The UV radiation may be selected from the group comprising UVA (wavelength between about 315 nm and about 400 nm), UVB (wavelength between about 280 and about 315 nm), UVC (wavelength between 100 nm and 280 nm). Photochemical active agents may be selected from a group comprising psoralens, e.g., amotosalen and riboflavin. In certain embodiments, the plasma may be processed by the INTERCEPT system as is known to a person skilled in the art and described throughout literature (Update on pathogen inactivation treatment of plasma, with the INTERCEPT Blood System: Current position on methodological, clinical and regulatory aspects. Irsch J., Transfus. Apher. Sci., 2015).

The term “S/D plasma” encompasses plasma comprising a reduced concentration or activity of Plasmin Inhibitor, such as Plasmin Inhibitor level equal to or less than 0.60 IU/ml or equal to or less than 0.50 IU/ml, for example Plasmin Inhibitor level between 0.20 and 0.30 IU/ml, more specifically between 0.22 and 0.25 IU/ml.

When compared with fresh frozen plasma (FFP), S/D plasma may comprise a reduced amount and/or activity of one or more of plasmin inhibitor, protein S, Factor XI, Factor V, Factor VIII, Factor X, a2 antiplasmin, anti-trypsin, von Willebrand factor (vWF), and von Willebrand factor-cleaving protease (VWFCP) also known as disintegrin and metalloproteinase with a thrombospondin type 1 motif, member 13 (ADAMTS-13), tumor necrosis factor-alpha (TNFα), interleukin-8 (IL-8), interleukin-10 (IL-10) (Benjamin and McLaughlin, 2012, Svae et al., 2007; Beeck and Hellstern, 1998; Doyle et al., 2003; Mast et al., 1999, Theusinger et al., 2011) and/or may comprise an increased amount and/or activity of Factor VII (Doyle et al., 2003).

The plasma, such as the S/D plasma, may be heat inactivated as known in the art, particularly to remove the complement. Where the present pharmaceutical formulations employ plasma, such as S/D plasma, autologous to the subject to be treated, it may be unnecessary to heat inactivate the plasma, such as the S/D plasma. Where the plasma, such as the S/D plasma, is at least partly allogeneic to the subject to be treated, it may be advantageous to heat inactivate the plasma, such as the S/D plasma. The plasma, such as the S/D plasma, may be autologous to the subject to be treated.

The term “autologous” with reference to plasma, such as the S/D plasma, denotes that the plasma, such as the S/D plasma, is obtained from the same subject to be contacted or treated with the plasma, such as the S/D plasma. Alternatively or additionally, the plasma, such as the S/D plasma, may also be “homologous” or “allogeneic” to the subject to be treated, i.e., obtained from one or more (pooled) subjects other than the subject to be contacted or treated with the plasma, such as the S/D plasma. Advantageously, allogeneic plasma, such as the S/D plasma, is commercially available and hence is an unrestricted source of plasma. In certain embodiments, the plasma, such as the S/D plasma, may be derived from warm-blooded animals, such as mammalian animals, such as humans.

In certain embodiments, the one or more plasmatic proteins may belong to the non-limiting group comprising of: albumin, globulin, fibrinogen, regulatory proteins and clotting factors. In further embodiments, the plasma proteins may be one or more of the following: prealbumin (transthyretin), alpha 1 antitrypsin, alpha 1 acid glycoprotein, alpha 1 fetoprotein, alpha 2 macroglobulin, gamma globulins, beta 2 microglobulin, haptoglobulin, ceruloplasmin, complement component 3, complement component 4, C-reactive protein (CRP), lipoproteins (chylomicrons, high-density lipoprotein, low-density lipoprotein and very-low-density lipoprotein), transferrin, prothrombin, Mannose-binding lectin, mannan-binding lectin (MBL) or mannan-binding protein (MBP). In further embodiments, the naturally occurring composition of plasma proteins may be maintained as such and used as component in the pharmaceutical formulation. Plasma compositions and concentration ranges of plasmatic proteins are well known to a person skilled in the art. In further embodiments, one plasmatic protein or a group of plasmatic proteins may have been separated from a collection of plasmatic proteins to be included in the formulation. In further embodiments, one plasmatic protein or a group of plasmatic proteins may have been separated from a collection of plasmatic proteins to be excluded from the pharmaceutical formulation.

The plasma or plasmatic proteins may be derived from a single blood donor. In further embodiments, the plasma or plasmatic proteins can be derived from a mixture of plasmatic proteins of at least two donors. In further embodiments, the plasma may be supplemented by additional proteins. In further embodiments, one or more plasmatic proteins contain post-translational modifications. In even further embodiments, the post-translational modification to one or more plasmatic proteins have been introduced after separation from the cellular components of the plasma. In further embodiments, the relative concentration of at least one plasmatic protein has been altered prior or after separation from the cellular components. In certain embodiments, plasmatic proteins are derived from blood donors belonging to a certain age. In further embodiment, plasmatic proteins are derived from blood donors with a known genotype. In further embodiments, the plasmatic proteins are derived from serum or comprise serum proteins. For example, the serum may be allogeneic or autologous with respect to the subject receiving the formulation. Preferably, the serum may be human serum, such that pharmaceutical formulations further comprising human serum are particularly suited for administration to human subjects. In certain embodiments, the serum may be obtained from solvent/detergent-treated plasma. The S/D plasma may be suitably treated to counter the action of the anticoagulant, such as to allow for conversion of fibrinogen into fibrin and the formation of the clot. In certain embodiments, the serum may be derived from warm-blooded animals, such as mammalian animals, such as humans.

In embodiments, the lyophilized pharmaceutical formulation comprises lyophilized serum. Hence, in particular aspects or embodiments, the lyophilized pharmaceutical formulation comprises lyophilized serum and hyaluronic acid or a derivative thereof, wherein the formulation, when reconstituted in an aqueous solution, has a reconstitution time of 15 minutes or less, preferably 10 minutes or less, more preferably 5 minutes or less.

Accordingly, an aspect relates to a lyophilized pharmaceutical formulation comprising lyophilized plasma and/or lyophilized serum, and hyaluronic acid or a derivative thereof, wherein the formulation, when reconstituted in an aqueous solution, has a reconstitution time of 15 minutes or less, preferably 10 minutes or less, more preferably 5 minutes or less. In embodiments, the formulation is configured for injection.

In certain embodiments, the formulation comprises from about 70% to about 99.9% by weight of lyophilized plasma and/or lyophilized serum. In certain embodiments, the formulation comprises from about 70% to about 99% by weight of lyophilized plasma and/or lyophilized serum, preferably from about 75% to about 99% by weight or from about 80% to about 97% by weight of lyophilized plasma and/or lyophilized serum. For instance, the formulation comprises from about 70% to about 95% by weight or from about 70% to about 90% by weight of lyophilized plasma and/or lyophilized serum.

In certain embodiments, the lyophilized serum comprises plasmatic proteins or derivatives thereof.

In certain embodiments, the formulation comprises at least about 30% by weight of plasmatic proteins or derivatives thereof, such as at least about 40% by weight, at least about 50% by weight, at least about 60%, at least about 70% by weight, at least about 80% by weight, or at least about 90% by weight of plasmatic proteins or derivatives thereof. In certain embodiments, the formulation comprises from about 30% to about 90% by weight of plasmatic proteins or derivatives thereof. In certain embodiments, the formulation comprises from about 30% to about 80% by weight of plasmatic proteins or derivatives thereof, from about 40% to about 75% by weight, particularly from about 50% to about 70% by weight, or from about 55% to about 60% by weight of plasmatic proteins or derivatives thereof. For instance, the formulation comprises from about 40% to about 70% by weight or from about 45% to about 65% by weight of plasmatic proteins or derivatives thereof.

Preferably, the plasmatic proteins are solvent/detergent-treated (S/D) plasma proteins, particularly human S/D plasma proteins. In certain embodiments, the plasmatic proteins or derivatives thereof are comprised in the pharmaceutical formulation at a weight percentage from about 30 wt % to about 80 wt %, preferably from about 40 wt % to about 75 wt %, more preferably from about 50 wt % to about 70 wt %, such as from about 55 wt % to about 65 wt %.

The terms “hyaluronic acid” or “HA” may be used interchangeably with “hyaluronan”, “hyaluronate” or “sodium hyaluronate”. The term “hyaluronic acid” refers to an anionic, non-sulfated polymer of disaccharides composed of D-glucuronic acid and N-acetyl-D-glucosamine, linked via alternating f3-1,4 and β-1,3 glycosidic bonds. Hyaluronic acid and derivatives belong to the group of glycosaminoglycans. In particular, the lyophilized pharmaceutical formulation comprises hyaluronic acid fibers or a derivative thereof.

The term “glycosaminoglycan” or “mucopolysaccharides” refer to unbranched polar polysaccharides consisting of a repeating disaccharide unit. Because of their water attracting properties, they may function as lubricant or shock absorber. As used herein, a lubricant functions by reducing friction between surfaces in mutual contact.

In certain embodiments, the derivative of hyaluronic acid is a salt of hyaluronic acid, an ester of hyaluronic acid with an alcohol of the aliphatic, heterocyclic or cycloaliphatic series, or a sulphated form of hyaluronic acid.

Hyaluronic acid derivatives include but are not limited to salts of hyaluronate such as sodium hyaluronate or an ester of hyaluronic acid with an alcohol of the aliphatic, heterocyclic or cycloaliphatic series, or a sulphated form of hyaluronic acid or combination of agents comprising hyaluronic acid. Without limitation, suitable derivatives may be salts of hyaluronic acid, such as preferably sodium hyaluronate.

In certain embodiments of the formulation or process as taught herein, the hyaluronic acid or derivative thereof comprises, consists essentially of, or consists of fibers having a molecular weight from 0.2 MDa to 4.5 MDa, preferably from 0.5 MDa to 1.5 MDa or from 0.5 MDa to 1.2 MDa.

The terms “(relative) molecular mass” and “molecular weight” may be used interchangeably herein and refer to the mass of a molecule.

In particular embodiments, the hyaluronic acid or derivative thereof may have a molecular mass ranging from about 0.2 MDa to about 8 MDa or more, such as ranging from about 0.2 MDa to about 6 MDa or ranging from about 0.4 MDa to about 6 MDa. In other particular embodiments, the hyaluronic acid or derivative thereof may have a molecular mass ranging from 0.2 MDa to about 4.5 MDa or ranging from about 0.4 MDa to about 4.5 MDa. In yet other particular embodiments, the hyaluronic acid or derivative thereof may have a molecular mass ranging from about 0.2 MDa to about 2.0 MDa, more in particular ranging from 0.4 MDa to about 1.5 MDa, even more in particular ranging from about 0.5 MDa to about 1.2 MDa. In certain embodiments, the hyaluronic acid or derivative thereof may have a molecular mass ranging from about 0.6 MDa to about 1.0 MDa. Advantageously, lyophilized pharmaceutical formulations comprising hyaluronic acid or a derivative thereof having a molecular mass ranging from about 0.5 MDa to about 1.2 MDa, preferably from about 0.6 MDa to about 1.0 MDa, allow to obtain a homogenous formulation for injection when reconstituted. In addition, the reconstituted formulations have satisfying viscosity for injection and provide sufficient viscosity in situ after administration.

Accordingly, a further aspect provides a lyophilized pharmaceutical formulation comprising lyophilized plasma and hyaluronic acid or a derivative thereof, wherein the hyaluronic acid or derivative thereof comprises fibers having a molecular weight from 0.5 MDa to 1.2 MDa, wherein the formulation, when reconstituted in an aqueous solution, has a reconstitution time of 15 minutes or less. Preferably, the hyaluronic acid or derivative thereof comprises fibers having a molecular weight from 0.6 MDa to 1.0 MDa. Such lyophilized pharmaceutical formulations allow to obtain a homogenous formulation for injection after reconstitution.

Preferably, the HA or derivative thereof have a low polydispersity index (PDI), which is a measure of the uniformity of the polymer population, or, stated differently, the distribution of molecular weights in a polymer population, and is calculated by the ratio of the weight average to the number average molecular weight of the polymer, as known by the skilled person. More in particular, the HA or derivative thereof has a polydispersity index of about 1.50 or less, such as about 1.40 or less, about 1.30 or less, about 1.20 or less, or 1.10 or less.

In certain embodiments, a single polymer form of hyaluronic acid or derivative thereof is used. In further embodiments, different lengths of hyaluronic acid or derivative thereof may be used in various relative concentrations in a preferred formulation. In further embodiments, different derivatives of hyaluronic acids are present in the formulation. In certain embodiment, the hyaluronic acid or derivative thereof is modified during the preparation of the pharmaceutical formulation.

In certain embodiments, hyaluronic acid may be present in the pharmaceutical formulation in combination with at least one hyaluronic acid derivative. Combinations of hyaluronic acid and derivatives may include but are not limited to hyaluronic acid and e.g. hyaluronic acid salt, e.g. hyaluronic acid ester, e.g. an alcohol of the aliphatic, e.g. heterocyclic or cycloaliphatic series of hyaluronic acid, e.g. any sulphated form of hyaluronic acid. In certain embodiments more than two hyaluronic acid derivatives may be present in the pharmaceutical formulation.

Further intended are hyaluronic acid derivatives that bind to any hyaluronic acid cell receptors including but by no means limited to CD44 receptor, receptor for HA-mediated motility (RHAMM) and intercellular adhesion molecule-1 (ICAM-1).

In certain embodiments, the lyophilized pharmaceutical formulation corresponding to one administration dose comprises from 1 mg to 100 mg of the hyaluronic acid or derivative thereof. For instance, the formulation corresponding to one administration dose may comprise from 2 mg to 90 mg, or from 5 to 75 mg of the hyaluronic acid or derivative thereof, preferably from 2 mg to 50 mg of the hyaluronic acid or derivative thereof, more preferably from 5 mg to 45 mg, from 5 mg to 40 mg, from 5 mg to 35 mg from, 5 mg to 30 mg or from 5 mg to 25 mg of the hyaluronic acid or derivative thereof.

In certain embodiments, the lyophilized pharmaceutical formulation comprises from about 5.0% to about 20.0% by weight of the hyaluronic acid or derivative thereof. For instance, the lyophilized pharmaceutical formulation comprises from about 7.5% to about 15.0% by weight or from about 10.0% to about 12.5% by weight of the hyaluronic acid or derivative thereof.

In certain embodiments, the lyophilized pharmaceutical formulation comprises at least one additional glycosaminoglycan wherein the glycosaminoglycan is selected from the group consisting of hyaluronic acid and derivatives thereof, a proteoglycan and derivatives thereof, a chondroitin sulfate, a keratan sulfate, a chitosan and derivatives thereof, a chitin and derivatives thereof. In further embodiments more than one additional glycosaminoglycan can be present in the formulation.

The term “chondroitin sulfate” refers to a polymer of disaccharides composed of N-acetylgalactosamine and glucuronic acid, each of which may be sulfated in variable positions and quantities. The chondroitin sulfate may be selected from chondroitin-4-sulfate, chondroitin-6-sulfate, chondroitin-2,6-sulfate, chondroitin-4,6-sulfate.

The lyophilized formulation as envisaged herein is typically a pale white-yellow cake. The lyophilized formulation as envisaged herein is a sterile cake.

The “reconstitution time” as used herein refers to the time between the moment when an aqueous solution is added to the lyophilized formulation (e.g. added above, inside or below the lyophilized formulation) and when a homogenous reconstituted product is obtained. The reconstitution is preferably performed by adding an aqueous solution to the lyophilized formulation, waiting until hydration of the lyophilized formulation, and thereafter mixing of the rehydrated formulation to obtain a homogenized reconstituted product. Mixing may be performed by rolling the vial (e.g. between the hands or mechanically) or by shaking the vial up and down (e.g. by hand or mechanically). Preferably, the lyophilized formulation is hydrated when all or substantially all, such as at least 90%, at least 95%, at least 96%, at least 97%, at least 98%, at least 99%, at least 99.5% or 100% of the lyophilized formulation has absorbed the aqueous solution.

In certain embodiments, reconstitution is obtained by mixing the lyophilized formulation with an aqueous solution such as water suitable for injection. In further embodiments, reconstitution may be promoted by agitating the solvent-lyophilized formulation mixture, such as by stirring, shaking, decanting, flipping, inverting, or rotating the vial comprising the pharmaceutical formulation. In further embodiments, reconstitution takes place immediately prior to administration of the formulation to a patient. In even further preferred embodiments, at least one additional manipulation precedes administration. In yet a further embodiment, reconstitution is at least partially obtained in a syringe.

Without limitation, the reconstitution time of the lyophilized pharmaceutical formulation may be about 15 minutes (min) or less, about 12 min or less, about 10 min or less, about 8 min or less, about 7 min or less, about 6 min or less, about 5 min or less, about 4.5 min or less, about 4 min or less, about 3.5 min or less, about 3 min or less, about 2.5 min or less, about 2 min or less, about 1.5 min or less, or about 1 min or less. Reconstitution time is referred herein as the time between the moment of adding an aqueous solution to the lyophilized formulation and the moment in time where the complete lyophilized product is dissolved as concluded by assessment with either the naked eye or impedance measurements. In further embodiments, the reconstitution time may be further improved by physical agitation of the vial comprising the pharmaceutical formulation.

In certain embodiments, the reconstitution time of the lyophilized pharmaceutical formulation may be from about 2 seconds to about 15 minutes, from about 10 seconds to about 15 minutes, from about 30 seconds to about 10 minutes, from about 1 minute to about 8 minutes, from about 2 minutes to about 8 minutes, from about 4 minutes to about 8 minutes, or from about 4 minutes to about 6 minutes.

In certain embodiments, the lyophilized pharmaceutical formulation has a density between 0.04 g/cm³ and 0.08 g/cm³, or between 0.05 g/cm³ and 0.07 g/cm³, such as e.g. a density of 0.062 g/cm³.

The density of the lyophilized pharmaceutical formulation may be determined (e.g. calculated) by dividing the weight of the lyophilized pharmaceutical formulation by the volume of the lyophilized pharmaceutical formulation. The weight may be calculated by subtraction of the weight of empty vial weight from the weight of the vial containing the lyophilized cake. The volume may be determined by measuring the dimensions of the lyophilized cake and calculating the volume. For instance, the lyophilized cake may have a cylindrical shape, and the volume may be determined by measuring the height and the diameter of the lyophilized cake, and calculating the volume.

In embodiments, the hyaluronic acid or derivative thereof may have a molecular mass ranging from about 0.5 MDa to about 1.2 MDa, and the lyophilized pharmaceutical formulation may have a density between 0.04 g/cm³ and 0.08 g/cm³. In certain embodiments, the hyaluronic acid or derivative thereof may have a molecular mass ranging from about 0.6 MDa to about 1.0 MDa, and the lyophilized pharmaceutical formulation may have a density between 0.04 g/cm³ and 0.08 g/cm³. In certain embodiments, the lyophilized pharmaceutical formulation comprises from about 30% to about 80% by weight of plasmatic proteins and from about 5.0% to about 20.0% by weight of the hyaluronic acid or derivative thereof, the hyaluronic acid or derivative thereof has a molecular mass ranging from about 0.5 MDa to about 1.2 MDa, and the lyophilized pharmaceutical formulation has a density between 0.04 g/cm³ and 0.08 g/cm³. In certain embodiments, the lyophilized pharmaceutical formulation comprises from about 30% to about 80% by weight of plasmatic proteins and from about 5.0% to about 20.0% by weight of the hyaluronic acid or derivative thereof, the hyaluronic acid or derivative thereof has a molecular mass ranging from about 0.6 MDa to about 1.0 MDa, and the lyophilized pharmaceutical formulation has a density between 0.04 g/cm³ and 0.08 g/cm³. Such lyophilized pharmaceutical formulations have a satisfying reconstitution time, e.g. a reconstitution time of about 15 minutes (min) or less, about 10 min or less, about 5 min or less, or about 2 min or less, while at the same time having a viscosity after reconstitution which both allows easy administration by injection and provides sufficient lubricating action after administration.

Hence, an aspect relates to a lyophilized pharmaceutical formulation comprising lyophilized plasma and hyaluronic acid or a derivative thereof, wherein the hyaluronic acid or derivative thereof has a molecular mass ranging from about 0.5 MDa to about 1.2 MDa, in particular a molecular mass ranging from about 0.6 MDa to about 1.0 MDa, and the formulation has a density between 0.04 g/cm³ and 0.08 g/cm³. In embodiments, the formulation comprises from about 30% to about 80% by weight of plasmatic proteins and from about 5.0% to about 20.0% by weight of the hyaluronic acid or derivative thereof.

In certain embodiments, the percentage of residual moisture of the formulation after lyophilisation is about 5.0% or less, about 4.0% or less, about 3.0% or less, about 2.5% or less.

Upon reconstitution of the lyophilized formulation as envisaged herein, the reconstituted formulation is a yellow, sterile, non-pyrogenic, viscoelastic homogenous solution. The term “non-pyrogenic” refers to the absence of fever-inducing or heat producing properties of the formulation. “Viscoelastic” or “viscoelasticity” is the property of materials that exhibit both viscous and elastic characteristics when undergoing deformation. In certain embodiment, the reconstituted pharmaceutical formulation may be further characterized by a viscosity of about 100 cP or more, about 200 cP or more, about 250 cP or more, such as between 200 cP and 500 cP or between 250 cP and 400 cP. Such viscosity advantageously allows easy administration by injection, while providing sufficient lubricating action after administration.

Accordingly, a further aspect provides a lyophilized pharmaceutical formulation comprising lyophilized plasma and hyaluronic acid or a derivative thereof, wherein the formulation, when reconstituted in an aqueous solution, has a reconstitution time of 15 minutes or less, and wherein the reconstituted pharmaceutical formulation is characterized by a viscosity of between 200 cP and 500 cP; preferably by a viscosity of between 250 cP and 400 cP. Such lyophilized pharmaceutical formulations, after reconstitution, advantageously allow easy administration by injection, while providing sufficient lubricating action after administration.

The term “viscosity” refers to a measure of the resistance of a fluid to deformation at a given rate.

The viscosity may be determined by a viscometer. For example, the viscosity may be assessed using a microVISC™ viscometer (RheoSense, CA, USA), according to the method of the supplier. For instance, a sensor cartridge, e.g. HB02, may be placed into the viscometer. Then, the sample may be loaded into a disposable pipette which is further mounted on the viscometer. The advanced parameters may be one or more of: Shear Rate=111.6 s⁻¹; Measuring volume=30 μl; Priming volume=15 μl; Pause time=5 s; and Range of sensor=60 to 5000 cP. As temperature is a well-known parameter that influences viscosity, each measure preferably has to be performed at 25.0±0.1° C. Before each use of the microVISC™, the viscosity of a reference oil may be measured to assess the calibration of the equipment.

The measuring chip may contain a rectangular slit flow channel constructed of borosilicate glass, with a uniform cross-sectional area. The sample may be injected at a constant flow rate though the flow channel where multiple pressure sensors mounted within the base monitor the pressure drop from the inlet to the outlet. The pressure drop may be correlated with the shear-stress at the boundary wall. The shear rate and shear stress may be directly related to the geometry of the rectangular slit and the flow rate which allow for the viscosity measurement. For example, a VROC® chip may assess the viscosity by measuring the pressure drop as a liquid flows through its rectangular slit microfluidic channel. The viscosity data may be exported into the microVISC™ control 2.0 software.

In embodiments, the lyophilized pharmaceutical formulation may be reconstituted in the aqueous solution at about 10 ml to about 14 ml of the aqueous solution per gram of the formulation. In embodiments, the lyophilized pharmaceutical formulation may be reconstituted in the aqueous solution at about 11 ml to about 13 ml of the aqueous solution per gram of the formulation. For instance, the lyophilized pharmaceutical formulation may be reconstituted in the aqueous solution at about 12 ml of the aqueous solution per gram of the formulation. A unit dose of the lyophilized pharmaceutical formulation (e.g. typically about 190 mg to about 230 mg) may typically be reconstituted in a volume of 2.4 ml of an aqueous solution. After reconstitution of the lyophilized formulation, the viscosity of the reconstituted formulation may be determined.

In embodiments, the lyophilized pharmaceutical formulation, when reconstituted in an aqueous solution at about 10 ml to about 14 ml of the aqueous solution per gram of the formulation, is characterized by a viscosity of between 200 cP and 500 cP; preferably by a viscosity of between 250 cP and 400 cP. In embodiments, the lyophilized pharmaceutical formulation, when reconstituted in an aqueous solution at about 11 ml to about 13 ml of the aqueous solution per gram of the formulation, is characterized by a viscosity of between 200 cP and 500 cP; preferably by a viscosity of between 250 cP and 400 cP. In embodiments, the lyophilized pharmaceutical formulation, when reconstituted in an aqueous solution at about 12 ml of the aqueous solution per gram of the formulation, is characterized by a viscosity of between 200 cP and 500 cP; preferably by a viscosity of between 250 cP and 400 cP.

In certain embodiments, the pharmaceutical formulation may be characterized by osmolality of about 200 milliosmol (mOsm)/kg or more, about 220 mOsm/kg or more, about 240 mOsm/kg or more, about 260 mOsm/kg or more, about 280 mOsm/kg or more, or about 300 mOsm/kg or more.

In certain embodiments, the lyophilized formulation further comprises an alpha-2 adrenergic receptor agonist, preferably wherein the alpha-2 adrenergic receptor agonist is clonidine or a derivative thereof.

In particular embodiments, the lyophilized pharmaceutical formulation further comprises an alpha-2 adrenergic receptor agonist, preferably wherein the alpha-2 adrenergic receptor agonist is selected from the group consisting of clonidine and derivatives thereof.

The term “alpha-2 adrenergic receptor agonist” or “α-2 adrenergic receptor agonist” refers to agents that mediate inhibition of adenylyl cyclase activity. Alpha-2 adrenergic receptor agonists are at least partially selective for the alpha-2 adrenergic receptor. In certain embodiments, the alpha-2 adrenergic receptor may not be the sole target of the agent. In further embodiments, the pharmaceutical formulation contains more than one alpha-2 adrenergic receptor agonist. In yet further embodiments, the different alpha-2 adrenergic receptor agonists have a synergistic effect. In yet further embodiments, the alpha-2 adrenergic receptor agonist is a synthetic compound with improved affinity for the alpha-2 adrenergic receptor compared to any natural alpha-2 adrenergic receptor ligand. In certain embodiments, the alpha-2 adrenergic receptor agonist engages in a covalent interaction with the alpha-2 adrenergic receptor. In yet further embodiments, the alpha-2 adrenergic receptor agonist does not physically interact with the alpha-2 adrenergic receptor and/or functions by interacting with natural alpha-2 adrenergic receptor ligands and/or influencing their cellular expression level. Alpha-2 adrenergic receptor agonists reduce pain through analgesic and anti-inflammatory effects. “Analgesic” as defined herein refers to pain killing, pain reducing or pain relieving properties. Analgesic components or compounds are used to achieve analgesia, the relief from pain.

In certain embodiments, the alpha-2 adrenergic receptor agonist may be selected from the group consisting of clonidine and derivatives thereof, including 2,6-dimethylclonidine, 4-azidoclonidine, 4-carboxyclonidine-methyl 3,5-dichlorotyrosine, 4-hydroxyclonidine, 4-iodoclonidine, alinidine, apraclonidine, chlorethylclonidine, clonidine 4-isothiocyanate, clonidine 4-methylisothiocyanate, clonidine receptor, clonidine-displacing substance, hydroxyphenacetyl aminoclonidine, N,N′-dimethylclonidine, p-aminoclonidine, and tiamenidine; imidazolidines, including imidazolines, impromidine, detomidine, medetomidine, dexmedetomidine, levamisole, losartane, lofexidine, miconazole, naphazoline, niridazole, nitroimidazoles, ondansetron, oxymetazoline, phentolamine, tetramisole, thiamazole, tizanidine, tolazoline, trimetaphan; imidazoles, including 4-(3-butoxy-4-méthoxybenzyl) imidazolidin-2-one, urocanic acid, amino-imidazole carboxamide, antazoline, biotine, bis (4-methyl-1-homo piperazinylthiocarbonyl) disulfide, carbimazole, cimetidine, clotrimazole, creatinine, dacarbazine, dexmedetomidine, econazole, enoximone, ethymizol, etomidate, fadrozole, fluspirilene, idazoxan, mivazerol; guanidines, including agmatine, betanidine, biguanides, cimetidine, creatine, gabexate, guanethidine, guanethidine sulfate, guanclofine, guanfacine, guanidine, guanoxabenz, impromidine, iodo-3 benzylguanidine, methylguanidine, mitoguazone, nitrosoguanidines, pinacidil, robenidine, sulfaguanidine, zanamivir; alpha-methyinorepherine, azepexole, 5-bromo-6-(2 imidazolidine-2-ylamino) quinoxalin, formoterol fumarate, indoramin, 6-allyl-2-amino-5,6,7,8-tetrahydro4H-thiazolo [4,5-d]azepine diHCl, nicergoline, rilmenidine, and xylazine.

In certain embodiments, the lyophilized pharmaceutical formulation may contain clonidine. In certain embodiments, the lyophilized pharmaceutical formulation comprises clonidine and at least one clonidine derivative. In further embodiments, the clonidine may be added to the formulation or be present in the formulation as clonidine HCl.

In yet further embodiments, clonidine is present in the formulation as one or more of the non-limiting group comprising: Arkamin, Aruclonin, Atensina, Catapin, Catapres, Catapresan, Catapressan, Chianda, Chlofazoline, Chlophazolin, Clonid-Ophtal, Clonidin, Clonidina, Clonidinã, Clonidine, Clonidine hydrochloride, Clonidinhydrochlorid, Clonidini, Clonidinum, Clonigen, Clonistada, Clonnirit, Clophelinum, Dixarit, Duraclon, Edolglau, Haemiton, Hypodine, Hypolax, Iporel, Isoglaucon, Jenloga, Kapvay, Klofelino, Kochaniin, Melzin, Menograine, Normopresan, Paracefan, Pinsanidine, Run Rui, and Winpress.

In certain embodiments, the lyophilized pharmaceutical formulation corresponding to one administration dose may comprise from 1 μg to 500 μg of the alpha-2-adrenergic receptor agonist, or from 25 to 400 μg, or from 50 to 250 μg, of the alpha-2-adrenergic receptor agonist. In certain embodiments, the lyophilized formulation may comprise from 50 μg to 150 μg, e.g., about 60 μg, about 70 μg, about 80 μg, about 90 μg, about 100 μg, about 110 μg, or about 120 μg of the alpha-2-adrenergic receptor agonist. Preferably, the formulation corresponding to one administration dose comprises from 2 μg to 250 μg of the alpha-2-adrenergic receptor agonist, more preferably from 5 μg to 125 μg of the alpha-2-adrenergic receptor agonist.

In certain embodiments, the formulation corresponding to one administration dose may comprise:
from 1 mg to 100 mg of the hyaluronic acid or derivative thereof, preferably from 2 mg to 50 mg of the hyaluronic acid or derivative thereof, more preferably from 5 mg to 40 mg of the hyaluronic acid or derivative thereof; and
optionally from 1 μg to 500 μg of the alpha-2-adrenergic receptor agonist, preferably from 2 μg to 250 μg of the alpha-2-adrenergic receptor agonist, more preferably from 5 μg to 125 μg of the alpha-2-adrenergic receptor agonist.

In certain embodiments, the lyophilized pharmaceutical formulation may comprise from about 0.01% to about 0.1% by weight of an alpha-2-adrenergic receptor agonist, such as clonidine or a derivative thereof. For instance, the lyophilized pharmaceutical formulation may comprise from about 0.05% to about 0.1% by weight of an alpha-2-adrenergic receptor agonist, such as clonidine or a derivative thereof.

In certain embodiments, the lyophilized pharmaceutical formulation further comprises at least one salt. In certain embodiments, the salt is a calcium salt. In certain embodiments, the salt may be calcium (di)chloride (CaCl₂).

Ca²⁺ may be added to the present pharmaceutical compositions, for example to enhance their coagulation and/or gellification in situ (e.g., where Ca²⁺ concentration found at the site of administration is found or expected to be inadequate to facilitate alone the coagulation/gellification of the compositions), or to achieve some degree of coagulation/gellification in vitro prior or after administration (e.g., to improve the injection capacity and/or integrity of the product). In such embodiments, Ca²⁺ may be typically added in the pharmaceutical composition at a concentration between about 0.1 and 5 wt %, preferably between about 0.5 wt % and about 3.0 wt %, more preferably between about 0.5 wt % and 2.0 wt % (as calcium vis-à-vis the total weight of the formulation).

Ca²⁺ may be suitably included in the pharmaceutical compositions through addition therein of a suitable amount of pharmaceutically acceptable calcium salt(s), preferably soluble calcium salt(s). Such Ca²⁺ salts may be formed with inorganic or organic acids. Examples of such salts include calcium (di)chloride (CaCl₂), calcium glycerophosphate, calcium phosphate, calcium hydrogen carbonate, calcium citrate, calcium sulphate, calcium lactate, calcium gluconate, calcium ascorbate, and mixtures thereof. Particularly preferred is CaCl₂, which displays advantageously good solubility and is well-tolerated in injectable solutions.

Pharmaceutical formulations corresponding to one administration dose intended herein may include between about 1 mg and about 10 mg CaCl₂, preferably between about 2 mg to 8 mg, preferably between about 3 mg and about 7 mg of CaCl₂. In certain embodiments, products intended for intra-articular or peri-articular administration may include between about 1 mg and about 10 mg CaCl₂, preferably between about 2 mg and about 7 mg, more preferably about 5 mg CaCl₂. In certain other embodiments, products intended for intra-osseous or pen-osseous administration may include between about 1 mg and about 10 mg CaCl₂, preferably between about 2 mg and about 7 mg, more preferably about 5 mg CaCl₂.

In certain embodiments, the lyophilized pharmaceutical formulation may comprise from about 1.5% to about 3.0% by weight of the salt, in particular a calcium salt, such as calcium chloride. For instance, the lyophilized pharmaceutical formulation may comprise from about 2.0% to about 3.0% by weight by weight of the salt in particular a calcium salt, such as calcium chloride.

In certain embodiments, the lyophilized pharmaceutical formulation further comprises at least one buffer solution comprising a weak acid and its conjugated base or vice versa (i.e., weak base and its conjugated acid) to buffer the pH of the composition.

In certain embodiments, the lyophilized pharmaceutical formulation further comprises at least one buffer component, in particular a buffer component configured for safe use in pharmaceutical applications. In certain embodiments, the buffer may be an acidic buffer. In alternative embodiments, the buffer may be a basic buffer. In yet further alternative embodiments, the buffer may be a phosphate buffer such as phosphate buffered saline (PBS).

In certain embodiments, the lyophilized pharmaceutical formulation may comprise from about 0.1% to about 2.0% by weight of the buffer component. For instance, the lyophilized pharmaceutical formulation may comprise from about 0.5% to about 1.0% by weight of the buffer component.

In certain embodiments, the buffer component may be selected from the non-limiting group of examples comprising 4-(cyclohexylamino)-1-butanesulfonic acid (CABS), N-cyclohexyl-3-aminopropanesulfonic acid (CAPS), 2-amino-2-methyl-1-propanol (AMP), N-cyclohexyl-2-hydroxyl-3-aminopropanesulfonic acid (CAPSO), N-cyclohexyl-2-aminoethanesulfonic acid (CHES), N-(1,1-dimethyl-2-hydroxyethyl)-3-amino-2-hydroxypropanesulfonic acid (AMPSO), N-tris(hydroxymethyl)methyl-4-aminobutanesulfonic acid (TABS), 2-Amino-2-methyl-1,3-propanediol (AMPD), [tris(hydroxymethyl)methylamino]propanesulfonic acid (TAPS), N-(2-hydroxyethyl)piperazine-N′-(4-butanesulfonic acid) (HEPBS), 2-(Bis(2-hydroxyethyl)amino)acetic acid (bicine), N-(2-hydroxy-1,1-bis(hydroxymethyl)ethyl)glycine (tricine), 3-[4-(2-hydroxyethyl)piperazin-1-yl]propane-1-sulfonic acid (EPPS), triethanolamine (TEA), Piperazine-1,4-bis(2-hydroxypropane sulfonic acid) dihydrate (POPSO), N-(Hydroxyethyl)piperazine-N′-2-hydroxypropanesulfonic acid (HEPPSO), tris(hydroxymethyl)aminomethane (trizma), 3-[[1,3-dihydroxy-2-(hydroxymethyl)propan-2-yl]amino]-2-hydroxypropane-1-sulfonic acid (TAPSO), 4-(N-morpholino)butanesulfonic acid (MOBS), 3-bis(2-hydroxyethyl) amino-2-hydroxypropane-1-sulfonic acid (DIPSO), 4-(2-hydroxyethyl)-1-piperazineethanesulfonic acid (HEPES), 2-[[1,3-dihydroxy-2-(hydroxymethyl)propan-2-yl]amino]ethanesulfonic acid (TES), 3-(N-morpholino)propanesulfonic acid (MOPS), 2-[bis(2-hydroxyethyl)amino]ethanesulfonic acid (BES), bis-tris propane (BTP), 3-(N-morpholino)hydroxypropanesulfonic acid (MOPSO), piperazine-N,N′-bis(2-ethanesulfonic acid) (PIPES), N-(2-Acetamido)-2-aminoethanesulfonic acid (ACES), 2-[(2-amino-2-oxoethyl)-(carboxymethyl)amino]acetic acid (ADA), 2-[Bis(2-hydroxyethyl)amino]-2-(hydroxymethyl)propane-1,3-diol (Bis-Tris), 2-(N-morpholino)ethanesulfonic acid (MES). Compositions of these buffer solutions and their preparation methods are described in the art and are therefore known to a person skilled in the art.

In certain embodiments, the buffer component is replaced by an acidic component such as hydrochloric acid (HCl).

In certain embodiments, the lyophilized pharmaceutical formulation further comprises at least one acidic component.

In certain embodiments, the acidic component is hydrochloric acid (HCl).

In certain embodiments, the lyophilized pharmaceutical formulation may comprise from about 0.1% to about 2.0% by weight of the acidic component, such as HCl. For instance, the lyophilized pharmaceutical formulation may comprise from about 0.5% to about 1.0% by weight of the acidic component, such as HCl.

In embodiments, the formulation may further comprise at least one salt, preferably wherein the salt is a calcium salt, more preferably wherein the salt is calcium chloride; and/or may further comprise at least one buffer component or acidic component, preferably wherein the acidic component is hydrochloric acid

In certain embodiments, the lyophilized pharmaceutical formulation comprises S/D plasma proteins and hyaluronic acid. In certain embodiments, the lyophilized pharmaceutical formulation comprises S/D plasma proteins, hyaluronic acid, and clonidine or a derivative thereof. In certain embodiments, the lyophilized pharmaceutical formulation comprises S/D plasma proteins, hyaluronic acid, and optionally clonidine or a derivative thereof, calcium (di)chloride, and/or hydrochloric acid.

In certain embodiments, the lyophilized pharmaceutical formulation comprises S/D plasma proteins, hyaluronic acid, clonidine or a derivative thereof, calcium (di)chloride, and hydrochloric acid.

In certain embodiments, the lyophilized pharmaceutical formulation comprises from about 30% to about 80% by weight of plasmatic proteins or derivatives thereof; and from about 5.0% to about 20.0% by weight of hyaluronic acid or a derivative thereof. In certain embodiments, the lyophilized pharmaceutical formulation comprises from about 30% to about 80% by weight of plasmatic proteins or derivatives thereof; from about 5.0% to about 20.0% by weight of hyaluronic acid or a derivative thereof; and from about 0.01% to about 0.1% by weight of an alpha-2-adrenergic receptor agonist.

In certain embodiments, the lyophilized pharmaceutical formulation comprises from about 30% to about 80% by weight of the plasmatic proteins or derivatives thereof; and from about 5.0% to about 20.0% by weight of the hyaluronic acid or derivative thereof; and optionally from about 0.01% to about 0.1% by weight of the alpha-2-adrenergic receptor agonist; from about 1.5% to about 3.0% by weight of the salt; and/or from about 0.1% to about 2.0% by weight of the buffer component or acidic component. In certain embodiments, the lyophilized pharmaceutical formulation comprises: from about 40% to about 75% by weight of the plasmatic proteins or derivatives thereof; and from about 5.0% to about 20.0% by weight of the hyaluronic acid or derivative thereof; and optionally from about 0.01% to about 0.1% by weight of the alpha-2-adrenergic receptor agonist; from about 1.5% to about 3.0% by weight of the salt; and/or from about 0.1% to about 2.0% by weight of the buffer component or acidic component. In certain embodiments, the lyophilized pharmaceutical formulation comprises from about 40% to about 75% by weight of the plasmatic proteins or derivatives thereof; and from about 10.0% to about 12.5% by weight of the hyaluronic acid or derivative thereof; and optionally from about 0.05% to about 0.1% by weight of the alpha-2-adrenergic receptor agonist; from about 2.0% to about 3.0% by weight of the salt; and/or from about 0.5% to about 1.0% by weight of the buffer component or acidic component. In certain embodiments, the lyophilized pharmaceutical formulation comprises from about 50% to about 70% by weight of the plasmatic proteins or derivatives thereof; and from about 10.0% to about 12.5% by weight of the hyaluronic acid or derivative thereof; and optionally from about 0.05% to about 0.1% by weight of the alpha-2-adrenergic receptor agonist; from about 2.0% to about 3.0% by weight of the salt; and/or from about 0.5% to about 1.0% by weight of the buffer component or acidic component.

In certain embodiments, the lyophilized pharmaceutical formulation comprises from about 30% to about 80% by weight of the plasmatic proteins or derivatives thereof; from about 5.0% to about 20.0% by weight of the hyaluronic acid or derivative thereof; from about 0.01% to about 0.1% by weight of the alpha-2-adrenergic receptor agonist; from about 1.5% to about 3.0% by weight of the salt; and from about 0.1% to about 2.0% by weight of the buffer component or acidic component.

In particular embodiments, the lyophilized formulation according to the present invention comprises between 30 wt % to about 80 wt % of plasmatic proteins or derivatives thereof, between 5.0 and 20.0 wt % of hyaluronic acid or a derivative thereof, and preferably between 0.01 and 0.1 wt % of an alpha-2-adrenergic receptor agonist as envisaged herein, preferably clonidine or a clonidine derivative; and/or between 1.0 and 5.0 wt % of a salt, preferably a calcium salt, as envisaged herein, more preferably calcium chloride, with wt % vis-à-vis the total weight of the lyophilized formulation. More in particular, the lyophilized formulation comprises between 30 wt % to 70 wt % of plasmatic proteins or derivatives thereof, between 5.0 and 15.0 wt % of hyaluronic acid or a derivative thereof, and preferably between 0.05 and 0.1 wt % of an alpha-2-adrenergic receptor agonist as envisaged herein and/or between 1.5 and 3.0 wt % of a salt, preferably a calcium salt, as envisaged herein, with wt % vis-à-vis the total weight of the lyophilized formulation.

In other embodiments, the lyophilized pharmaceutical formulation further comprises or may be co-administered with one or more further pharmaceutical active ingredients.

“pharmaceutical active ingredient” or “API” as referred to herein is to be interpreted according to the definition of the term by the World Health organization: a substance used in a finished pharmaceutical product (FPP), intended to furnish pharmacological activity or to otherwise have direct effect in the diagnosis, cure, mitigation, treatment or prevention of disease, or to have direct effect in restoring, correcting or modifying physiological functions in human beings.

In certain embodiments, at least one active pharmaceutical ingredient is added to the formulation prior to lyophilisation. In this case, the release of each active ingredient may be identical or different such as for instance in case of a combination of two active ingredients in which the first one is presented as an immediate release form and the second one as a controlled release. Similarly, a combination of immediate release and controlled release form may also be obtained for the same active ingredient, in order to provide a rapid and sustained effect. In further embodiments, at least one active pharmaceutical ingredient is added during reconstitution. In yet further embodiments, the additional active pharmaceutical formulation is added immediately prior to administration to the patient. In certain embodiments, the pharmaceutical formulation comprises at least two additional pharmaceutical active ingredients. In further embodiments, the different additional pharmaceutical active ingredients are added at different points in time during manufacturing of the pharmaceutical formulation.

In further embodiments, the lyophilized pharmaceutical formulation further comprises or may be co-administered with one or more further pharmaceutical active ingredients wherein the one or more pharmaceutical active ingredient is, each independently, selected from the group consisting of: a cell composition, a pharmaceutical active compound, a protein, a peptide, and a small organic molecule.

The applicability of the present invention is not limited to any pharmaceutical active ingredient or class of pharmaceutical active ingredients. The pharmaceutical active ingredient may be pharmacologically active itself, or may be converted into a pharmacologically active species by a chemical or enzymatic process in the body, i.e., the pharmaceutical active ingredient may be a prodrug. The present pharmaceutical formulations may be particularly useful for poorly-stable pharmaceutical active ingredients. Illustrative non-limiting examples of poorly-stable pharmaceutical active ingredients include peptides and proteins such as growth factors, peptide-like active ingredients, antibodies and vaccines, small interfering RNA (siRNA), DNA, hormones, etc.

The term “growth factor” as used herein refers to a biologically active substance which influences proliferation, growth, differentiation, survival and/or migration of various cell types, and may affect developmental, morphological and functional changes in an organism, either alone or when modulated by other substances. A growth factor may typically act by binding, as a ligand, to a receptor (e.g. surface or intracellular receptor) present in cells responsive to the growth factor. A growth factor herein may be particularly a proteinaceous entity comprising one or more polypeptide chains By means of example and not limitation, the term “growth factor” encompasses the members of the fibroblast growth factor (FGF) family, bone morphogenetic protein (BMP) family, platelet-derived growth factor (PDGF) family, transforming growth factor beta (TGFβ) family, nerve growth factor (NGF) family, epidermal growth factor (EGF) family, insulin-like growth factor (IGF) family, growth differentiation factor (GDF) family, hepatocyte growth factor (HGF) family, hematopoietic growth factors (HeGFs), platelet-derived endothelial cell growth factor (PD-ECGF), angiopoietin, vascular endothelial growth factor (VEGF) family, glucocorticoids, and the like.

The term “pharmaceutical active ingredient” also encompasses any pharmacologically active salts, esters, N-oxides or prodrugs of the title compound or substance.

In particular, the lyophilized pharmaceutical formulation may further comprise one or more substance with osteogenic or chondrogenic, osteo or chondro-inductive and/or osteo or chondro-conductive properties. In preferred embodiments, such substance may be selected from the group comprising or consisting of a fibroblast growth factor (FGF), preferably FGF-2, a transforming growth factor beta (TGFB), preferably TGFB-1, platelet-derived growth factor (PDGF), interleukin-8 (IL-8), a bone morphogenetic protein (BMP), for example any one or more of BMP-2, BMP-4, BMP-6 and BMP-7, parathyroid hormone (PTH), parathyroid hormone-related protein (PTHrp), VEGF and stem cell factor (SCF). Any one such substance may be included in a pharmaceutical composition at a concentration sufficient to achieve its desired osteogenic, osteo-inductive and/or osteo-conductive effect(s) when administered to a subject, while insofar possible avoiding unwanted side effects.

Typically but without limitation, any one such substance may be comprised in the pharmaceutical formulation at a concentration between 0.01 ng/mg and 1 mg/mg, for example 0.1 ng/mg to 100 μg/mg, for example 1 ng/mg to 50 μg/mg.

The term “osteo-inductive” refers to the capacity of a component such as a peptide growth factor to recruit immature cells such as stem cells, MSC and stimulate those cells to differentiate into pre-osteoblasts and mature osteoblasts, thereby forming bone tissue. The present pharmaceutical compositions may further comprise a component with osteo-inductive properties such as an osteo-inductive protein or peptide, for instance a bone morphogenetic protein, such as BMP-2, BMP-7 or BMP-4; a hydrogel or biopolymer such as hyaluronic acid or derivatives thereof, collagen, fibrinogen, osteonectin, or osteocalcin. Preferably, the pharmaceutical compositions may further comprise hyaluronic acid or derivatives thereof, collagen or fibrinogen.

The term “osteo-conductive” refers to the ability of a component to serve as a scaffold on which bone cells can attach, migrate, grow and produce new bone. The pharmaceutical compositions may further comprise a component with osteo-conductive properties, for example, an osteo-conductive scaffold or matrix or surface such as without limitation tricalcium phosphate, hydroxyapatite, combination of hydroxyapatite/tricalcium phosphate particles (HA/TCP), gelatine, poly-lactic acid, poly-lactic glycolic acid, hyaluronic acid, chitosan, poly-L-lysine, or collagen.

The pharmaceutical formulations according to the present invention may further include or be co-administered with a complementary bioactive factor or osteo-inductive protein such as a bone morphogenetic protein, such as BMP-2, BMP-7 or BMP-4, or any other growth factor. Other potential accompanying components include inorganic sources of calcium or phosphate suitable for assisting bone regeneration (WO 00/07639). If desired, cell preparation can be administered on a carrier matrix or material to provide improved tissue regeneration. For example, the material can be a hydrogel, or a biopolymer such as gelatine, collagen, hyaluronic acid or derivatives thereof, osteonectin, fibrinogen, or osteocalcin. Biomaterials can be synthesized according to standard techniques (e.g., Mikos et al., Biomaterials 14:323, 1993; Mikos et al., Polymer 35:1068, 1994; Cook et al., J. Biomed. Mater. Res. 35:513, 1997).

Typically, the lyophilized pharmaceutical formulation is mixed with at least one aqueous solution prior to administration, preferably wherein the aqueous solution is water for injection.

“Water for injection”, “aqua ad iniectabilia”, “aqua ad injectionem”, “WFI” or “aqua ad ini.” as defined herein refers to water without any significant contamination suitable for injection to a person. As defined herein, the water is considered sterile and/or other substances are added to make the solution about isotonic. In certain embodiments, the aqueous solution may be a physiological saline or isotonic saline solution.

Saline is a mixture of sodium chloride in water and has a numerous uses in medicine known to a person skilled in the art. A common saline solution contains about 9 grams of sterile salt per liter solution. In certain embodiments, the amount of salt per liter may be different.

In certain embodiments additional active pharmaceutical ingredients are added to the aqueous solution prior to mixing with the lyophilized pharmaceutical formulation. In further embodiments, the aqueous solution contains at least one pharmaceutic excipient. In further embodiments, the aqueous solution may have a temperature from about 10° C. to about 37° C.

In certain embodiments, the aqueous solution used to reconstitute the lyophilized pharmaceutical formulation may comprise biological material. By means of guidance and not limitation this biological material may be a cell composition that may comprise mesenchymal stem cells (MSC), osteoprogenitors, osteoblastic cells, osteocytes, chondroblastic cells, and/or chondrocytes. The pharmaceutical formulation thus allows for delivery of such cell composition. This viscous quality of the present pharmaceutical formulations can ensure localised delivery of and suitable supportive environment for the delivered cells.

The term “mesenchymal stem cell” or “MSC”, as used herein, refers to an adult, mesoderm-derived stem cell that is capable of generating cells of mesenchymal lineages, typically of two or more mesenchymal lineages, e.g., osteocytic (bone), chondrocytic (cartilage), myocytic (muscle), tendonocytic (tendon), fibroblastic (connective tissue), adipocytic (fat) and stromogenic (marrow stroma) lineage. MSC may be isolated from, e.g., bone marrow, trabecular bone, blood, umbilical cord, placenta, foetal yolk sac, skin (dermis), specifically foetal and adolescent skin, periosteum and adipose tissue. Human MSC, their isolation, in vitro expansion, and differentiation, have been described in, e.g., U.S. Pat. Nos. 5,486,359; 5,811,094; 5,736,396; 5,837,539; or U.S. Pat. No. 5,827,740. Any MSC described in the art and isolated by any method described in the art may be suitable in the present pharmaceutical formulations.

The term MSC also encompasses the progeny of MSC, e.g., progeny obtained by in vitro or ex vivo proliferation (propagation) of MSC obtained from a biological sample of an animal or human subject.

Preferable MSC have the potential of generating cells of at least the osteogenic (bone) lineage, such as, e.g., osteoprogenitors and/or pre-osteoblasts and/or osteoblasts and/or osteocytes, etc. or of at least the chondrogenic (cartilage) lineage, such as, e.g., chondrogenic cells and/or chondroblasts and/or chondrocytes, etc.

The term “stem cell” refers generally to an unspecialized or relatively less specialized and proliferation-competent cell, which is capable of self-renewal, i.e., can proliferate without differentiation, and which or the progeny of which can give rise to at least one relatively more specialized cell type. The term encompasses stem cells capable of substantially unlimited self-renewal, i.e., wherein the progeny of a stem cell or at least part thereof substantially retains the unspecialized or relatively less specialized phenotype, the differentiation potential, and the proliferation capacity of the mother stem cell, as well as stem cells which display limited self-renewal, i.e., wherein the capacity of the progeny or part thereof for further proliferation and/or differentiation is demonstrably reduced compared to the mother cell. By means of example and not limitation, a stem cell may give rise to descendants that can differentiate along one or more lineages to produce increasingly relatively more specialized cells, wherein such descendants and/or increasingly relatively more specialized cells may themselves be stem cells as defined herein, or even to produce terminally differentiated cells, i.e., fully specialized cells, which may be post-mitotic.

The term “adult stem cell” as used herein refers to a stem cell present in or obtained from (such as isolated from) an organism at the foetal stage or after birth, such as for example after achieving adulthood.

As used herein, “osteoprogenitors” may particularly comprise early and late osteoprogenitors. “Osteoblastic cells” may particularly encompass pre-osteoblasts, osteoblasts and osteocytes, and the term may more preferably denote pre-osteoblasts and osteoblasts. All these terms are well-known per se and as used herein may typically refer to cells having an osteogenic phenotype, and that can contribute to, or are capable of developing to cells which can contribute to, the formation of bone material or bone matrix.

By means of further guidance and not limitation, osteoprogenitors and osteoblastic cells, as well as cell populations comprising osteoprogenitors and/or osteoblastic cells may display the following characteristics:

a) the cells comprise expression of Runx2, a multifunctional transcription factor that regulates osteoblast differentiation and the expression of many extracellular matrix protein genes during osteoblast differentiation;

b) the cells comprise expression of at least one of the following: alkaline phosphatase (ALP), more specifically ALP of the bone-liver-kidney type; and more preferably also comprise expression of one or more additional bone markers such as osteocalcin (OCN), procollagen type 1 amino-terminal propeptide (P1NP), osteonectin (ON), osteopontin (OP) and/or bone sialoprotein (BSP), and/or one or more additional bone matrix proteins such as decorin and/or osteoprotegerin (OPG);

c) the cells substantially do not express CD45 (e.g., less than about 10%, preferably less than about 5%, more preferably less than about 2% of the cells may express CD45);

d) the cells show evidence of ability to mineralize the external surroundings, or synthesize calcium-containing extracellular matrix (e.g., when exposed to osteogenic medium; see Jaiswal et al. J Cell Biochem, 1997, vol. 64, 295-312). Calcium accumulation inside cells and deposition into matrix proteins can be conventionally measured for example by culturing in ‘Ca’, washing and re-culturing, and then determining any radioactivity present inside the cell or deposited into the extracellular matrix (U.S. Pat. No. 5,972,703), or using an Alizarin red-based mineralization assay (see, e.g., Gregory et al. Analytical Biochemistry, 2004, vol. 329, 77-84);

e) the cells substantially do not differentiate towards neither of cells of adipocytic lineage (e.g., adipocytes) or chondrocytic lineage (e.g., chondrocytes). The absence of differentiation towards such cell lineages may be tested using standard differentiation inducing conditions established in the art (e.g., see Pittenger et al. Science, 1999, vol. 284, 143-7), and assaying methods (e.g., when induced, adipocytes typically stain with oil red 0 showing lipid accumulation; chondrocytes typically stain with alcian blue or safranin 0). Substantially lacking propensity towards adipogenic and/or chondrogenic differentiation may typically mean that less than 20%, or less than 10%, or less than 5%, or less than 1% of the tested cells would show signs of adipogenic or chondrogenic differentiation when applied to the respective test.

The cells may further comprise expression of one or more cell recruitment factors such as IL6 and/or VEGF.

As used herein, “chondroblastic cells” may particularly comprise chondroblasts, i.e., young (not matured, immature) cartilage cells active in the secretion of extracellular matrix. Chondroblasts are considered to arise by differentiation from mesenchymal stem cells. The term “chondrocyte” more specifically refers to a mature cartilage cell necessary for the maintenance of cartilaginous matrix. These terms are well-known per se and as used herein may typically refer to cells having a chondrogenic phenotype, and that can contribute to, or are capable of developing to cells which can contribute to, the formation of cartilage or cartilaginous matrix.

Wherein a cell is said to be positive for (or to express or comprise expression of) a particular marker, this means that a skilled person will conclude the presence or evidence of a distinct signal, e.g., antibody-detectable or detection by reverse transcription polymerase chain reaction, for that marker when carrying out the appropriate measurement, compared to suitable controls. Where the method allows for quantitative assessment of the marker, positive cells may on average generate a signal that is significantly different from the control, e.g., but without limitation, at least 1.5-fold higher than such signal generated by control cells, e.g., at least 2-fold, at least 4-fold, at least 10-fold, at least 20-fold, at least 30-fold, at least 40-fold, at least 50-fold higher or even higher.

The expression of the above cell-specific markers can be detected using any suitable immunological technique known in the art, such as immuno-cytochemistry or affinity adsorption, Western blot analysis, FACS, ELISA, etc., or by any suitable biochemical assay of enzyme activity (e.g., for ALP), or by any suitable technique of measuring the quantity of the marker mRNA, e.g., Northern blot, semi-quantitative or quantitative RT-PCR, etc. Sequence data for markers listed in this disclosure are known and can be obtained from public databases such as GenBank (http://www.ncbi.nlm.nih.gov/).

The cells of the cell composition may be animal cells, preferably warm-blooded animal cells, more preferably mammalian cells, such as human cells or non-human mammalian cells, and most preferably human cells. In certain embodiments, the pharmaceutical formulation is provided as a part in a kit-of-parts. The kit-of-parts may comprise the lyophilized pharmaceutical formulation as defined in any embodiment of this invention contained in one or more containers or storage vials, particularly with each vial corresponding to one treatment dose, a syringe comprising an aqueous solution. Preferably, the kit-of-parts further comprises at least one needle. The kit-of-parts may contain the pharmaceutical formulation as defined in any embodiment as described herein.

In certain embodiments, the amount of syringes and/or needles may be adjusted according to the amount of lyophilized pharmaceutical formulation contained in the storage vial. In certain embodiments, the kit may additionally comprise a disinfectant and/or anti-inflammatory component. The anti-inflammatory component may be selected from a group comprising a treatment fluid, a spray, a lotion, a cream, an ointment, a gel, a gum, a bandage, a dermal patch, a plaster. Anti-inflammatory components have been described throughout the state of the art. In further embodiments, the kit may comprise instructions to reconstitute the lyophilized formulation and/or instructions for administration. In yet a further embodiment, the lyophilized formulation is contained in a dual chamber syringe and is fully reconstituted in the syringe. In a further embodiment, the lyophilized formulation is contained in a multi-chamber syringe, such as a double syringe comprising the lyophilized pharmaceutical composition in one compartment and the aqueous solution in a second compartment.

In yet a further embodiment the kit-of-parts may comprise more than one vial. In a certain embodiment, the kit comprises vials with different lyophilized pharmaceutical formulations, wherein the hyaluronic acid or derivative thereof and/or the plasmatic proteins or derivatives thereof vary between the different formulations. In further embodiments, vials differ in that the lyophilized pharmaceutical formulation comprises different alpha-2 adrenergic receptor agonists and/or salt and/or buffer component or acidic component. In certain embodiments, the kit comprises an additional component that allows for testing the degree of reconstitution. In yet a further embodiment, the kit comprises at least one bandage, dermal patch, or plaster.

A further aspect relates to a process for preparing a lyophilized pharmaceutical formulation as taught herein, comprising the following steps:
(a) mixing plasmatic proteins or derivatives thereof, hyaluronic acid or derivative thereof, and an aqueous solution, thereby obtaining a bulk mixture having a concentration of the plasmatic proteins or derivatives thereof of 20 mg/ml to 50 mg/ml and a concentration of the hyaluronic acid or derivative thereof of 4 mg/ml to 8 mg/ml;
(b) sterilizing the bulk mixture by steam sterilization or filter sterilization, thereby obtaining a sterile mixture; and
(c) lyophilizing the sterile mixture, thereby obtaining the lyophilized pharmaceutical formulation.

Preferably, an aspect provides a process for preparing a lyophilized pharmaceutical formulation as defined herein, comprising the following steps:
(a) mixing plasma, such as S/D plasma, and hyaluronic acid or derivative thereof, thereby obtaining a bulk mixture having a concentration of plasmatic proteins of 20 mg/ml to 50 mg/ml and a concentration of the hyaluronic acid or derivative thereof of 4 mg/ml to 8 mg/ml;
(b) sterilizing the bulk mixture by steam sterilization or filter sterilization, thereby obtaining a sterile mixture; and
(c) lyophilizing the sterile mixture, thereby obtaining the lyophilized pharmaceutical formulation.

In another aspect, the invention provides a process or method for preparing a lyophilized pharmaceutical formulation, comprising the steps of
(a) mixing plasmatic proteins or derivatives thereof, preferably S/D plasma proteins, hyaluronic acid or derivative thereof and an aqueous solution, thereby obtaining a bulk mixture;
(b) sterilizing the bulk mixture thereby obtaining a sterile mixture; and
(c) lyophilizing the sterile mixture, thereby obtaining the lyophilized pharmaceutical formulation.

Accordingly, an aspect provides a process for preparing a lyophilized pharmaceutical formulation, comprising the following steps:
(a) mixing plasma, preferably S/D plasma, and hyaluronic acid or derivative thereof, thereby obtaining a bulk mixture;
(b) sterilizing the bulk mixture thereby obtaining a sterile mixture; and
(c) lyophilizing the sterile mixture, thereby obtaining the lyophilized pharmaceutical formulation.

Further, an aspect provides a process for preparing a lyophilized pharmaceutical formulation, comprising the following steps:
(a) mixing plasma, preferably S/D plasma, and hyaluronic acid or derivative thereof, thereby obtaining a bulk mixture;
(b) sterilizing the bulk mixture thereby obtaining a sterile mixture; and
(c) lyophilizing the sterile mixture, thereby obtaining the lyophilized pharmaceutical formulation;

wherein step (a) comprises the steps of (a1) dissolving the hyaluronic acid or derivative in an aqueous solution, thereby obtaining a first solution; (a2) preparing a second solution comprising the plasma, and, optionally, an alpha-2 adrenergic receptor agonist, and (a3) mixing the first and second solution to obtain the bulk mixture.

In embodiments, the bulk mixture has a concentration of plasmatic proteins of 20 mg/ml to 50 mg/ml and a concentration of the hyaluronic acid or derivative thereof of 4 mg/ml to 8 mg/ml.

The mixing of the hyaluronic acid or derivative thereof is typically obtained by stirring, shaking, decantating, flipping, inverting, agitating or rotating of the hyaluronic acid and/or derivative thereof with the aqueous solution. In embodiments, the first solution may comprise about 1.0 to 30 mg/ml hyaluronic acid or a derivative thereof, preferably about 2.0 to 20 mg/ml, more preferably about 4.0 to 16.0 mg/ml hyaluronic acid or a derivative thereof, such as about 8.0 to 12.0 mg/ml hyaluronic acid or a derivative thereof. The bulk mixture particularly comprises about 1.0 to 15 mg/ml hyaluronic acid or a derivative thereof, preferably about 2.0 to 10 mg/ml, more preferably about 4.0 to 8.0 mg/ml hyaluronic acid or a derivative thereof.

In certain embodiments, the plasmatic proteins, preferably S/D plasma proteins are provided as S/D plasma. Preferably, the bulk mixture comprises from about 70% to about 99.9% by weight of S/D plasma in the pharmaceutical formulation, preferably from about 75% to about 99%, or preferably from about 80% to about 97% by weight of S/D plasma. Preferably, the second solution comprises from about 70% to about 100% by weight of plasmatic proteins, such as S/D plasma proteins, preferably from about 75% to about 99%, or preferably from about 80% to about 97% by weight of plasmatic proteins, such as S/D plasma proteins. The bulk mixture particularly comprises between 20 mg/ml and 50 mg/ml plasmatic proteins or plasmatic protein derivatives.

In embodiments, the second solution may comprise from about 20% (v/v) to about 100% (v/v) of plasma, such as S/D plasma. For instance, the second solution may comprise from about 40% (v/v) to about 99% (v/v), from about 50% (v/v) to about 98% (v/v), from about 60% (v/v) to about 97% (v/v), from about 70% (v/v) to about 96% (v/v), or from about 80% (v/v) to about 95% (v/v) of plasma, such as S/D plasma.

In embodiments, the bulk mixture may comprise at most 50% (v/v) of plasma such as S/D plasma. In embodiments, the bulk mixture may comprise from about 10% (v/v) to about 50% (v/v) of plasma, such as S/D plasma. For instance, the bulk mixture may comprise from about 20% (v/v) to about 49% (v/v), from about 25% (v/v) to about 48% (v/v), from about 30% (v/v) to about 47% (v/v), from about 35% (v/v) to about 46% (v/v), or from about 40% (v/v) to about 45% (v/v) of plasma, such as S/D plasma.

In certain embodiments, the bulk mixture further comprises one or more of the following:
an alpha-2-adrenergic receptor agonist as described herein, preferably clonidine or a derivative thereof;
a salt, preferably a calcium salt, such as calcium dichloride; and/or
a buffer component or acidic component, preferably HCl.

Accordingly, in certain embodiments, step (a) further comprises the step of mixing an alpha-2 adrenergic receptor agonist, preferably clonidine, and/or a salt, preferably a calcium salt, and/or an buffer component or acidic component, preferably HCl, thereby obtaining a bulk mixture wherein the concentration of the alpha-2 adrenergic receptor agonist, preferably clonidine or clonidine derivative as envisaged herein, is between 20 μg/ml and 35 μg/ml and/or wherein the concentration of the salt, preferably a calcium salt, more preferably calcium dichloride, is between 0.5 mg/ml and 1.5 mg/ml.

In certain embodiments, step (a) may further comprise mixing an alpha-2 adrenergic receptor agonist, a salt, and/or a buffer component or acidic component, thereby obtaining a bulk mixture having a concentration of the alpha-2 adrenergic receptor agonist of 20 μg/ml to 35 μg/ml, a concentration of the salt of 0.5 mg/ml to 1.5 mg/ml, and/or a concentration of the buffer component or acidic component of 0.05 mg/ml to 3.0 mg/ml.

In certain embodiments, the bulk mixture further comprises one or more other components, including but not limited to pharmaceutical excipients, serum and/or other blood components, further active pharmaceutical ingredients selected from a group comprising a pharmaceutical active compound, a protein, a peptide, and a small organic molecule.

In certain embodiments, step (a) comprises the steps of (a1) dissolving the hyaluronic acid or derivative in an aqueous solution, thereby obtaining a first solution; (a2) preparing a second solution comprising the plasmatic proteins, and (a3) mixing the first and second solution to obtain the bulk mixture.

In certain embodiments, step (a) comprises the steps (a1) dissolving the hyaluronic acid or derivative in an aqueous solution, thereby obtaining a first solution; (a2) preparing a second solution comprising the plasmatic proteins, and an alpha-2 adrenergic receptor agonist, and (a3) mixing the first and second solution to obtain the bulk mixture.

In certain embodiments, step (a) comprises the steps of (a1) dissolving the hyaluronic acid or derivative in an aqueous solution, thereby obtaining a first solution; (a2) preparing a second solution comprising the plasma and/or serum, and (a3) mixing the first and second solution to obtain the bulk mixture.

In certain embodiments, step (a) comprises the steps of (a1) dissolving the hyaluronic acid or derivative in an aqueous solution, thereby obtaining a first solution; (a2) preparing a second solution comprising the plasma and/or serum, and an alpha-2 adrenergic receptor agonist, and (a3) mixing the first and second solution to obtain the bulk mixture.

In certain embodiments, step (a) comprises the step of dissolving the hyaluronic acid or derivative in an aqueous solution, thereby obtaining a first solution. In certain embodiments, the hyaluronic acid or derivative thereof is first dissolved in an aqueous solution, obtaining a first solution, prior to mixing with a second solution. The step of dissolving the hyaluronic acid or derivative in an aqueous solution may last for at least 10 hours, such as at least 12 hours, at least 14 hours, at least 16 hours, or at least 18 hours. This step allows complete hydration of the hyaluronic acid or derivative thereof.

In certain embodiments, step (a) comprises the step of preparing a second solution comprising the plasmatic proteins. In certain embodiments, step (a) comprises the step of preparing a second solution comprising the plasma and/or serum. In certain embodiments, the second solution further comprises additional components such as the non-limiting examples described above including an alpha-2-adrenergic receptor agonist as described herein, preferably clonidine or a derivative thereof, a salt, and an acidic component. Accordingly, in certain embodiments, the method comprises (a2) preparing a second solution comprising the plasmatic proteins, an alpha-2-adrenergic receptor agonist as described herein, preferably clonidine or a derivative thereof, a salt, and an acidic component. In certain embodiments, the first solution comprising hyaluronic acid or a derivative thereof is mixed with the second solution comprising: plasmatic proteins, preferably an S/D plasma; an 2-adrenergic receptor agonist as described herein, preferably clonidine or a derivative thereof; a salt; and an acidic component. In embodiments, the method comprises (a2) preparing a second solution comprising the plasma and/or serum, an alpha-2-adrenergic receptor agonist as described herein, preferably clonidine or a derivative thereof, a salt, and an acidic component. In certain embodiments, the first solution comprising hyaluronic acid or a derivative thereof is mixed with the second solution comprising: plasma and/or serum, preferably an S/D plasma; an 2-adrenergic receptor agonist as described herein, preferably clonidine or a derivative thereof; a salt; and an acidic component. The salt may be but is not restricted to be a calcium salt such as calcium dichloride (CaCl₂ or CaCl₂.2H₂O). The acidic component may be but is not restricted to be hydrogen chloride (HCl). It is understood that the above-described first and second solution are combined to obtain the bulk mixture.

In certain embodiments, step (a) comprises the step of mixing the first and second solution to obtain the bulk mixture. In certain embodiments, the first solution and the second solution are mixed in a ratio of at least 1:1 (v/v), such as in a ratio of 1.5:1, 2:1, 3:1, 4:1 (v/v) or more. In certain embodiments, the first solution and the second solution are mixed in a ratio of 1:1 (v/v). Thereby, the resulting lyophilized pharmaceutical formulation has satisfying density, hence allowing quick and homogenous reconstitution of the lyophilized pharmaceutical formulation.

The phrase “mixing the first solution and the second solution in a ratio of 1:1 (v/v)” refers to mixing equal volumes of the first solution and the second solution. Advantageously, equal volumes of the first and the second solution are mixed.

In certain embodiments, the method of sterilization is filter sterilization. As used herein, the term “filter sterilization”, “filtration sterilization”, or “microporous filtration” refers to a method having as goal the sterilization of a sample, mixture or formulation. In certain embodiments, a membrane is used to obtain filtration, allowing for exclusion of components and/or organisms based upon size.

Further intended are filtration methods wherein the filter material used may include nylon, polycarbonate, cellulose, acetate, polyvinylidene fluoride (PVDF), and polyethersulfone (PES). These materials are characterized by differences in protein retention, flow rate, and the presence of leachable materials.

In certain embodiments, the method of sterilization is sterilization by steam. Herein, the formulation is exposed to saturated steam at elevated temperatures, e.g. from about 121° C. to about 134° C. By means of guidance and not limitation, steam sterilization may be achieved by using an autoclave. In certain embodiments, the temperature for steam sterilization is from about 125 to about 130° C. In certain embodiments, different methods of sterilization may be combined. In further embodiments, the different methods of sterilization are performed in succession. In certain embodiments, the formulation is subjected to the steam sterilization conditions for about 3 to about 30 minutes. In certain embodiments, the formulation is subjected to the steam sterilization conditions for about 10 to about 20 minutes.

The term “autoclave” as used herein refers to a pressure chamber able to achieve elevated temperatures and pressures differing from atmospheric pressure. The sterilization times needed to achieve sterilization may vary depending on multiple parameters such as the amount and nature of the material that needs to be sterilized. It is known to a person skilled in the art that steam sterilization times may be inversely correlated to the used steam sterilization temperature.

By means of further guidance and not limitation, lyophilisation may be performed according to the following steps: filling individual sterile containers with aliquots of the bulk solution and partially stoppering the containers under aseptic conditions, transporting the partially stoppered containers to the lyophilizer and loading into the chamber under aseptic conditions, freezing the solution by placing the partially stoppered containers on cooled shelves in a lyophilisation chamber or pre-freezing in another chamber, applying a vacuum to the chamber and heating the shelves in order to evaporate the water from the frozen state, and finally complete stoppering of the vials by hydraulic or screw rod stoppering mechanisms that may be installed in the lyophilisation device. In certain embodiments, no partial stoppering is done on the samples at the start of the lyophilisation process and a complete stoppering is performed after lyophilisation.

The term “stopper” refers to the seal of a vial inhibiting the lyophilized formulation to escape the vial and/or allow a sterile environment inside to vial to be contained. Alternative terms may be caps, lids, seals, crimp seals, or any means that allows closing of the vial.

The step of lyophilisation is possible using a variety of parameters, repetitions thereof, by combination, or by additional steps. The temperature and/or duration of the single or multiple freeze steps in the lyophilisation process may be adjusted to obtain a specific size of ice crystals prior to sublimation. Drying phases are executed under reduced pressures that may range from about 0.1 mbar to 0.005 mbar or from about 0.1 mbar to about 0.01 mbar.

In further embodiments, the bulk mixture is aliquoted prior to lyophilisation so that the resulting vials contain an amount of the formulation corresponding to a single dose for administration. In alternative embodiments, aliquoting is performed after lyophilisation. In further embodiments, no aliquoting takes place and the resulting vial containing the pharmaceutical formulation corresponds to more than one administration doses. In yet further embodiments, the resulting vial contains a volume higher than a volume corresponding to a natural number of administration doses to anticipate for adhesive effects of the reconstituted formulation to the walls of the vial, syringe or stopper. In further embodiments, this additional volume may be about 20% of the volume needed for a natural number of administration doses, about 15% of the volume needed, about 10% of the volume needed, about 5% of the volume needed, about 2% of the volume needed, about 1% of the volume needed.

In certain embodiments the lyophilized pharmaceutical formulation may be obtainable or obtained by a process as defined herein.

A further aspect relates to the lyophilized pharmaceutical formulations obtainable or obtained by any embodiment of the processes described herein.

In certain embodiments, the lyophilized pharmaceutical formulation comprises hyaluronic acid or derivative thereof, plasmatic proteins and an alpha-2 adrenergic receptor agonist. In further embodiment, the lyophilized pharmaceutical formulation consists or consists mainly of hyaluronic acid, plasmatic proteins, clonidine, calcium chloride, and a buffer component or an acidic component such as hydrogen chloride. In particularly preferred embodiments, the pharmaceutical formulation comprises from about 30 wt % to about 80 wt % plasmatic proteins, comprises between 5.0 and 20.0 wt % hyaluronic acid fibers or derivative thereof with about a molecular weight from about 0.2 MDa to 4.5 MDa, particularly from about 0.5 MDa to about 1.2 MDa, has a density between about 0.04 and 0.08 mg/ml, and has a degree of swelling from about 9 to about 30, and further comprising between 0.01 and 0.1 wt % of clonidine or a clonidine derivative, and/or between 1.5 and 3.0 wt % of a calcium salt, particularly calcium chloride.

A related aspect concerns the lyophilized pharmaceutical formulation as described above for use as a medicament.

A related aspect concerns the lyophilized pharmaceutical formulation as described above for use in the treatment (including throughout the present specification therapeutic and/or preventative measures) of a musculoskeletal disease. Preferably, said musculoskeletal disease may be a bone disease or a joint disease.

A “joint”, “articulation” or “articular surface” as defined herein refers to a connection between bones in a body which link the skeletal system into a functional whole. Suitable joints for treatment using the pharmaceutical formulation can be selected from the group comprising monoarticular joints, oligoarticular or pauciarticular joints and polyarticular joints. Joints as defined herein may relate to one or more members of the functional classification group comprising fibrous joints, cartilaginous joints, synovial joints or facet joints. The joints may be selected from the group consisting of the joints of the hand, elbow joints, wrist joints, axillary articulations, sternoclavicular joints, vertebral articulations, temporomandibular joints, sacroiliac joints, hip joints, knee joints or articulations of the foot.

A further aspect provides a method of treating a musculoskeletal disease in a subject in need of such a treatment, comprising administering a therapeutically effective amount of a lyophilized pharmaceutical formulation as taught herein to the subject, wherein the the lyophilized pharmaceutical formulation is mixed with an aqueous solution prior to administration.

A further aspect provides the use of a lyophilized pharmaceutical formulation as taught herein for the manufacture of a medicament for the treatment of a musculoskeletal disease in a subject.

The term “musculoskeletal disease”, as used herein, refers to any type of bone disease, muscle disease, joint disease, or chondrodystrophy, the treatment of which may benefit from the administration of the present pharmaceutical formulation to a subject having the disease. The term also encompasses diseases affecting tendons and/or ligaments). In particular, such disease may be characterized, e.g., by decreased bone and/or cartilage formation or excessive bone and/or cartilage resorption, by decreased number, viability or function of osteoblasts or osteocytes present in the bone and/or chondroblast or chondrocytes present in the cartilage, decreased bone mass and/or cartilage mass in a subject, thinning of bone, compromised bone strength or elasticity, etc.

Non-limiting examples of musculoskeletal diseases may include local or systemic disorders, such as, any type of osteoporosis or osteopenia, e.g., primary, postmenopausal, senile, corticoid-induced, bisphosphonates-induced, and radiotherapy-induced; any secondary, mono- or multisite osteonecrosis; any type of fracture, e.g., non-union, mal-union, delayed union fractures or compression, conditions requiring bone fusion (e.g., spinal fusions and rebuilding), maxillo-facial fractures, congenital bone defect, bone reconstruction, e.g., after traumatic injury or cancer surgery, and cranio-facial bone reconstruction; traumatic arthritis, focal cartilage and/or joint defect, focal degenerative arthritis; osteoarthritis, degenerative arthritis, gonarthrosis, and coxarthrosis; osteogenesis imperfecta; osteolytic bone cancer; Paget's Disease, endocrinological disorders, hypophosphatemia, hypocalcemia, renal osteodystrophy, osteomalacia, adynamic bone disease, hyperparathyroidism, primary hyperparathyroidism, secondary hyperparathyroidism; periodontal disease; Gorham-Stout disease and McCune-Albright syndrome; rheumatoid arthritis; spondyloarthropathies, including ankylosing spondylitis, psoriatic arthritis, enteropathic arthropathy, and undifferentiated spondyloarthritis and reactive arthritis; systemic lupus erythematosus and related syndromes; scleroderma and related disorders; Sjogren's Syndrome; systemic vasculitis, including Giant cell arteritis (Horton's disease), Takayasu's arteritis, polymyalgia rheumatica, ANCA-associated vasculitis (such as Wegener's granulomatosis, microscopic polyangiitis, and Churg-Strauss Syndrome), Behcet's Syndrome, and other polyarteritis and related disorders (such as polyarteritis nodosa, Cogan's Syndrome, and Buerger's disease); arthritis accompanying other systemic inflammatory diseases, including amyloidosis and sarcoidosis; crystal arthropathies, including gout, calcium pyrophosphate dihydrate disease, disorders or syndromes associated with articular deposition of calcium phosphate or calcium oxalate crystals; chondrocalcinosis and neuropathic arthropathy; Felty's Syndrome and Reiter's Syndrome; Lyme disease and rheumatic fever.

In embodiments, the musculoskeletal disease may be osteoarthritis.

As used herein, a phrase such as “a subject in need of treatment” includes subjects that would benefit from treatment of a given condition, particularly a musculoskeletal disease. Such subjects may include, without limitation, those that have been diagnosed with said condition, those prone to develop said condition and/or those in who said condition is to be prevented.

The terms “treat” or “treatment” encompass both the therapeutic treatment of an already developed disease or condition, such as the therapy of an already developed musculoskeletal disease, as well as prophylactic or preventive measures, wherein the aim is to prevent or lessen the chances of incidence of an undesired affliction, such as to prevent occurrence, development and progression of a musculoskeletal disease. Beneficial or desired clinical results may include, without limitation, alleviation of one or more symptoms or one or more biological markers, diminishment of extent of disease, stabilized (i.e., not worsening) state of disease, delay or slowing of disease progression, amelioration or palliation of the disease state, and the like. “Treatment” can also mean prolonging survival as compared to expected survival if not receiving treatment.

The term “prophylactically effective amount” refers to an amount of an active compound or pharmaceutical agent that inhibits or delays in a subject the onset of a disorder as being sought by a researcher, veterinarian, medical doctor or other clinician.

The formulations and methods as taught herein allow to administer a therapeutically effective amount of a pharmaceutical active ingredients in subjects having a musculoskeletal disease which will benefit from such treatment. The term “therapeutically effective amount” as used herein, refers to an amount of active compound or pharmaceutical agent that elicits the biological or medicinal response in a subject that is being sought by a surgeon, researcher, veterinarian, medical doctor or other clinician, which may include inter alia alleviation of the symptoms of the disease or condition being treated.

Appropriate therapeutically effective doses of a pharmaceutical active compound or pharmaceutical active ingredient in the present formulation may be determined by a qualified physician with due regard to the nature of the pharmaceutical active compound or pharmaceutical active ingredient, the disease condition and severity, and the age, size and condition of the patient.

In certain embodiments, the musculoskeletal disease may affect tendons and/or ligaments. In certain embodiments, the pharmaceutical formulation as described herein may be part of a combinatorial therapy strategy not limited to e.g. other medicinal therapies known to a person skilled in the art, or e.g. kinesiotherapy. In further embodiments, the lyophilized formulation is used as measurement to prevent symptoms from arising. In further embodiments, the bone or joint disease may be classified as a progressive bone disease or progressive joint disease. In yet further embodiments, the bone disease or joint disease is a genetic disorder or disease. In even further embodiments, the bone disease or joint disease is an age-related disease. In even further embodiments, the purpose of using of the lyophilized formulation is merely symptomatic.

Further intended is a method for treating a musculoskeletal disease in a subject in need of such treatment, comprising administering to said subject a therapeutically or prophylactically effective amount of the pharmaceutical formulation as described above. In certain embodiments, the pharmaceutical formulation is administered at multiple points in time. In further embodiments, the different administrations are separated from each other by regular time intervals. In other embodiment, the time intervals between different administrations are increasing by a certain multiplicity. In yet other embodiments, the time intervals between different administrations are increasing exponentially. In certain embodiments, aliquots of one therapeutic dose are administered via separate injection entry positions.

The present application also provides aspects and embodiments as set forth in the following Statements:
Statement 1. A lyophilized pharmaceutical formulation comprising plasmatic proteins or derivatives thereof and hyaluronic acid or a derivative thereof, wherein the formulation, when reconstituted in an aqueous solution, has a reconstitution time of 15 minutes or less.
Statement 2. The lyophilized pharmaceutical formulation according to statement 1, wherein the formulation further comprises an alpha-2 adrenergic receptor agonist, preferably wherein the alpha-2 adrenergic receptor agonist is clonidine or a derivative thereof.
Statement 3. The lyophilized pharmaceutical formulation according to statement 1 or 2, wherein the formulation comprises from about 30% to about 80% by weight of the plasmatic proteins or derivatives thereof.
Statement 4. The lyophilized pharmaceutical formulation according to any one of statements 1 to 3, wherein the plasmatic proteins are solvent/detergent-treated (S/D) plasma proteins, preferably human S/D plasma proteins, and/or wherein the derivative of hyaluronic acid is a salt of hyaluronic acid, an ester of hyaluronic acid with an alcohol of the aliphatic, heterocyclic or cycloaliphatic series, or a sulphated form of hyaluronic acid.
Statement 5. The lyophilized pharmaceutical formulation according to any one of statements 1 to 4, wherein the hyaluronic acid or derivative thereof comprises fibers having a molecular weight from 0.2 MDa to 4.5 MDa, preferably from 0.5 MDa to 1.2 MDa.
Statement 6. The lyophilized pharmaceutical formulation according to any one of statements 1 to 5, further comprising at least one salt, preferably wherein the salt is a calcium salt, more preferably wherein the salt is calcium chloride; and/or further comprising at least one buffer component or acidic component, preferably wherein the acidic component is hydrochloric acid.
Statement 7. The lyophilized pharmaceutical formulation according to any one of statements 1 to 6, further comprising one or more pharmaceutical active ingredients.
Statement 8. The lyophilized pharmaceutical formulation according to statement 7, wherein the one or more pharmaceutical active ingredient is, each independently, selected from the group consisting of: a cell composition, a pharmaceutical active compound, a protein, a peptide, and a small organic molecule.
Statement 9. The lyophilized pharmaceutical formulation according to statement 8, wherein the cell composition comprises mesenchymal stem cells (MSC), osteoprogenitors, osteoblastic cells, osteocytes, chondroblastic cells, and/or chondrocytes.
Statement 10. The lyophilized pharmaceutical formulation according to statement 8, wherein the pharmaceutical active protein or peptide is a growth factor, preferably a growth factor selected from the group consisting of a fibroblast growth factor (FGF), a transforming growth factor beta (TGFB), platelet-derived growth factor (POGF), interleukin-8 (IL-8), a bone morpho-20 genetic protein (BMP), parathyroid hormone (PTH), parathyroid hormone-related protein (PTHrp), and stem cell factor (SCF); more preferably a growth factor selected from the group consisting of FGF-2, TGFB-1, POGF, IL-8, BMP-2, BMP-4, BMP-6, BMP-7, PTH, PTHrp, and SCF.
Statement 11. The lyophilized pharmaceutical formulation according to any one of statements 1 to 10, further comprising at least one glycosaminoglycan.
Statement 12. The lyophilized pharmaceutical formulation according to statement 11, wherein one of the additional glycosaminoglycans is a chondroitin sulfate.
Statement 13. The lyophilized pharmaceutical formulation according to any one of statements 1 to 12, further comprising serum, preferably human serum.
Statement 14. The lyophilized pharmaceutical formulation according to any one of statements 1 to 13, further comprising proteins of whole blood or proteins of a fractionated component of whole blood, preferably wherein the whole blood is human whole blood.
Statement 15. The lyophilized pharmaceutical formulation according to any of statements 1 to 14, wherein the formulation corresponding to one administration dose comprises:
   from 1 mg to 100 mg of the hyaluronic acid or derivative thereof, preferably from 2 mg to 50 mg of the hyaluronic acid or derivative thereof, more preferably from 5 mg to 40 mg of the hyaluronic acid or derivative thereof; and
   optionally from 1 μg to 500 μg of the alpha-2-adrenergic receptor agonist, preferably from 2 μg to 250 μg of the alpha-2-adrenergic receptor agonist, more preferably from 5 μg to 125 μg of the alpha-2-adrenergic receptor agonist.
Statement 16. The lyophilized pharmaceutical formulation according to any one of statements 1 to 15, wherein the formulation comprises:
   from about 30% to about 80% by weight of the plasmatic proteins or derivatives thereof; and
   from about 5.0% to about 20.0% by weight of the hyaluronic acid or derivative thereof; and optionally
   from about 0.01% to about 0.1% by weight of the alpha-2-adrenergic receptor agonist;
   from about 1.5% to about 3.0% by weight of the salt; and/or
   from about 0.1% to about 2.0% by weight of the buffer component or acidic component.
Statement 17. The lyophilized pharmaceutical formulation according to any one of statements 1 to 16, wherein the lyophilized formulation has a degree of swelling from 9 to 30.
Statement 18. The lyophilized pharmaceutical formulation according to any one of aspects 1 to 17, which is configured for parenteral administration after reconstitution, preferably for intra-osseous, peri-osseous, intra-articular, or peri-articular administration, or for intra-tendon, pen-tendon, intra-ligament or pen-ligament administration after reconstitution.
Statement 19. A kit-of-parts comprising:
   a lyophilized pharmaceutical formulation according to any one of statements 1 to 18;
   a syringe comprising an aqueous solution; and
   preferably, at least one needle.
Statement 20. A process for preparing a lyophilized pharmaceutical formulation according to any one of statements 1 to 18, comprising the following steps:
   (a) mixing plasmatic proteins or derivatives thereof, hyaluronic acid or derivative thereof, and an aqueous solution, thereby obtaining a bulk mixture having a concentration of the plasmatic proteins or derivatives thereof of 20 mg/ml to 50 mg/ml and a concentration of the hyaluronic acid or derivative thereof of 4 mg/ml to 8 mg/ml;
   (b) sterilizing the bulk mixture by steam sterilization or filter sterilization, thereby obtaining a sterile mixture; and
   (c) lyophilizing the sterile mixture, thereby obtaining the lyophilized pharmaceutical formulation.
Statement 21. The process according to statement 20, wherein step (a) further comprises mixing an alpha-2 adrenergic receptor agonist, a salt, and/or a buffer component or acidic component, thereby obtaining a bulk mixture having a concentration of the alpha-2 adrenergic receptor agonist of 20 μg/ml to 35 μg/ml, a concentration of the salt of 0.5 mg/ml to 1.5 mg/ml, and/or a concentration of the buffer component or acidic component of 0.05 mg/ml to 3.0 mg/ml.
Statement 22. The process according to statement 20 or 21, wherein the hyaluronic acid or derivative thereof comprises fibers having a molecular weight from 0.2 MDa to 4.5 MDa, preferably from 0.5 MDa to 1.2 MDa.
Statement 23. A lyophilized pharmaceutical formulation obtainable or obtained by a process according to any one of statements 20 to 22, preferably the lyophilized pharmaceutical formulation according to any one of statements 1 to 18 obtainable or obtained by a process according to any one of statements 20 to 22.
Statement 24. The lyophilized pharmaceutical formulation according to any one of statements 1 to 18 or 23, for use in the treatment of a musculoskeletal disease, preferably wherein the lyophilized pharmaceutical formulation is mixed with an aqueous solution prior to administration.
Statement 25. The lyophilized pharmaceutical formulation for use according to statement 24, preferably wherein the musculoskeletal disease is a bone disease or a joint disease.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The herein disclosed aspects and embodiments of the invention are further supported by the following non-limiting examples.

### EXAMPLES

### Example 1—Method for Obtaining a Lyophilized Pharmaceutical Composition According to an Embodiment of the Invention

A bulk mixture was prepared by mixing 5 grams HA fibers (0.6-1 MDa), 500 ml water, 1.25 ml clonidine HCl (20 mg/ml), 476.25 ml S/D plasma, 10 ml HCl (1M), and 12.5 ml CaCl₂ (80 mg/ml).

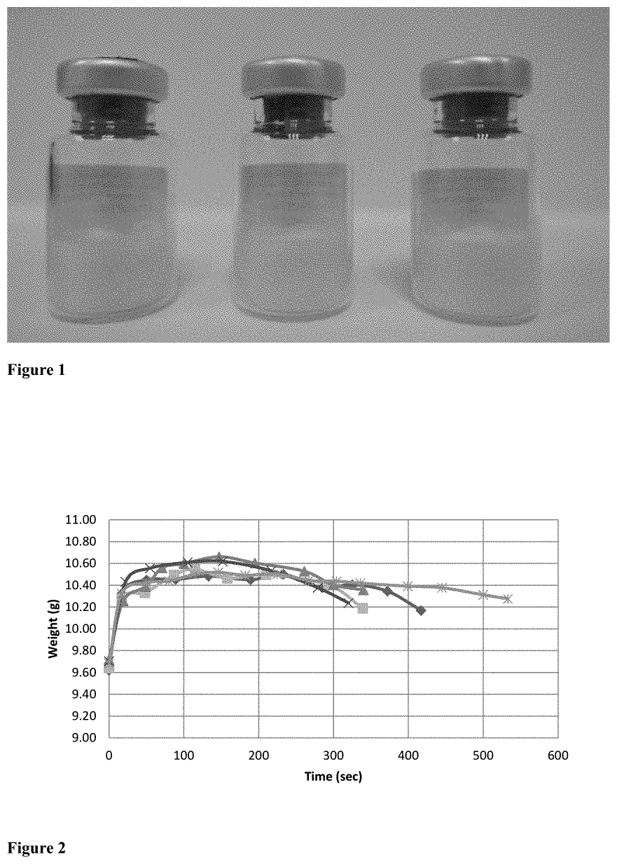

The resulting bulk mixture was sterilized by filtration. In a following step, storage vials were filled with 5 ml of the sterilized bulk mixture and subsequently lyophilized. After the lyophilisation phase, the vials were stoppered. FIG. 1 shows three vials comprising a lyophilized pharmaceutical formulation according to an embodiment of the invention. The lyophilized formulation was a pale white-yellow cake. Prior to injection to a patient in need thereof, the pharmaceutical formulation in the storage vials is reconstituted in 2.4 ml water.

### Example 2—Composition of a Reconstituted Lyophilized Pharmaceutical Formulation Corresponding to One Administration Dose According to Certain Embodiments

| | | |
|---|---|---|
| Hyaluronic acid | 25 | mg |
| S/D plasma | 188 | mg |
| (lyophilised S/D | | |
| plasma) | | |
| Clonidine | 125 | μg |
| CaCl₂ | 5 | mg |
| HCl | 1.9 | mg |

### Example 3—Characteristics of a Lyophilized Pharmaceutical Formulation Corresponding to Certain Embodiments

| | |
|---|---|
| Weight percentage protein material | 61% |
| Density | 0.062 |

### Example 4. Reconstitution Time of Different Formulations According to Certain Embodiments

4.1 Different Molecular Weight HA

Compositions with different molecular weight of HA (high molecular weight (MW): 3.5-4.5 MDa, medium MW: 0.6-1 MDa, low MW: less than 0.6 MDa) and different dilution factors of the hyaluronic acid (see 4.2 below) are produced.

4.2 Different Dilution of Bulk Mixture

Three different bulk mixtures were prepared: Hyaluronic acid fibers (1 g) were mixed with 95.25 ml S/D plasma, 5 mg clonidine HCl (0.25 ml of a 20 mg/ml solution), 200 mg CaCl₂ (2.5 ml of 80 mg/ml solution), 72.92 mg HCl (2 ml of a solution at 1M), to obtain a first bulk mixture (no dilution, referred to as “1×”).

Further, hyaluronic acid fibers (1 g) were mixed with 100 ml H₂O to obtain a first solution A. Hyaluronic acid fibers (1 g) were also mixed with 200 ml H₂O to obtain a first solution B. A second solution was prepared by mixing 95.25 ml S/D plasma, 5 mg clonidine HCl (0.25 ml of a 20 mg/ml solution), 200 mg CaCl₂ (2.5 ml of 80 mg/ml solution), and 72.92 mg HCl (2 ml of a solution at 1M). The first solution A (100 ml) was mixed with the second solution (100 ml) at a ratio of 1:1 (v/v) to obtain a bulk mixture referred to as “2×”. The first solution B (200 ml) was mixed with the second solution (100 ml) at a ratio of 2:1 (v/v) to obtain a bulk mixture referred to as “3×”.

4.3 Freeze-Dried Cake

Bulk mixtures for every HA MW and dilution are transferred to a vial for lyophilisation. Density of the lyophilized product is determined by the ratio cake weight/cake volume. The cake weight is obtained by the subtraction of the empty vial weight to the weight of vial containing the lyophilized cake. The cake volume is calculated using the formula: π×R²×h where R is the radius of the cake and h the cake height.

The density of the dried cake decreased according to increasing dilution of the bulk mixture before the freeze drying: density (1×)>density (2×)>density (3×) (Table 1). The lyophilized pharmaceutical formulations prepared by dilution had a density of between 0.04 g/cm³ and 0.08 g/cm³. The density was not impacted by the HA molecular weight (Table 1).

TABLE 1
Weight (g), volume (cm³) and density (g/cm³) of lyophilized pharmaceutical
formulations according to embodiments of the invention (n = 15);
Bulk mixture of the formulations before lyophilisation: 1x, 2x, or 3x

| | | HA Low MW | | | HA Medium MW | | | HA High MW | | |
|---|---|---|---|---|---|---|---|---|---|---|
| | | weight | volume | density | weight | volume | density | weight | volume | density |
| 1x | Mean | 0.194 | 1.856 | 0.105 | 0.197 | 2.013 | 0.098 | 0.198 | 1.775 | 0.111 |
| | SD | 0.013 | 0.039 | 0.009 | 0.018 | 0.073 | 0.008 | 0.006 | 0.025 | 0.004 |
| 2x | Mean | 0.199 | 3.488 | 0.057 | 0.190 | 3.431 | 0.055 | 0.194 | 3.205 | 0.061 |
| | SD | 0.009 | 0.177 | 0.004 | 0.004 | 0.144 | 0.002 | 0.004 | 0.071 | 0.001 |
| 3x | Mean | 0.188 | 4.059 | 0.046 | 0.199 | 4.572 | 0.043 | 0.194 | 4.293 | 0.045 |
| | SD | 0.006 | 0.169 | 0.002 | 0.006 | 0.156 | 0.002 | 0.004 | 0.148 | 0.002 |

The lyophilized pharmaceutical formulations prepared by a method involving dilution (e.g. bulk mixtures 2× and 3×) had an optimal density for reconstitution. In view thereof, the lyophilized pharmaceutical formulations prepared by a method comprising dilution (2× or 3×) were preferred in order to obtain a cake having satisfying density.

Cake Absorption Capacity

The absorption capacity of the different freeze-dried products was assessed by measuring the cake weight over time. Briefly, 9.6 ml water were added on freeze-dried cake ensuring its complete immersion. The extra water was then removed. The weight was measured just after having removed the extra water. The process was repeated several times.

Hydration curves representing the weight in function of time showed that the lyophilized pharmaceutical formulations according to embodiments of the invention were fully hydrated in 30 seconds. FIG. 2 provides a representative hydration curve illustrating the weight in function of time for five lyophilized pharmaceutical formulations according to an embodiment of the present invention prepared by mixing of the first solution and second solution at a ratio of 1:1 (v/v) (2×) and with medium molecular weight HA. The lyophilized pharmaceutical formulations were fully hydrated in less than 30 seconds (first time point after 0 sec was ranging from 17 sec to 22 sec).

When comparing the absorption capacity of the lyophilized pharmaceutical formulations according to the HA molecular weight (HA low, medium and high MW), the hydration capacity of lyophilized pharmaceutical formulations with medium and high MW HA was higher than the hydration capacity of lyophilized pharmaceutical formulations containing low MW HA (data not shown).

The hydration capacity of the lyophilized pharmaceutical formulations increased with increasing HA molecular weight: absorption capacity_{HA high MW}>absorption capacity_{HA medium MW}>absorption capacity_{HA low WV}. The hydration capacity increased with the density reduction of the lyophilized pharmaceutical formulations: absorption capacity_{3×}>absorption capacity_{2×}>absorption capacity_{1×}.

Reconstitution Time of the Dried Cake (in Water)

2.4 ml water for injection was added to each lyophilized sample using a syringe (dead volume of 0.1 ml). After the complete cake hydration, the vial was mixed between the hands by rolling (from 2 to 4 mixing run) or shaken up and down by hand. Mixing lasted only about 30 seconds. The reconstitution time was assessed using a timer. Reconstitution time started just after the water was added and stopped after the complete resuspension of the product. Reconstitution was assessed by visual inspection. Reconstituted product was a pale yellow to yellow, cloudy and slightly viscous solution. The solution sometimes contained bubbles due to shaking.

The data show that for high molecular weight HA, homogenous reconstitution was not possible for all lyophilized pharmaceutical formulations. The analysis of the reconstitution time was thus performed only for lyophilized pharmaceutical formulations containing low MW HA and medium MW HA.

TABLE 2
Hydration time (min:sec), mixing time (min:sec) and reconstitution time (min:sec)
of lyophilized pharmaceutical formulations according to embodiments of the invention
(n = 5); Bulk mixture of the formulations before lyophilisation: 1x, 2x, or 3x

| | | HA Low MW | | | HA Medium MW | | |
|---|---|---|---|---|---|---|---|
| | | Hydration | Mixing | Reconstitution | Hydration | Mixing | Reconstitution |
| | | time | time | time | time | time | time |
| 1x | Mean | 01:43 | 00:39 | 02:22 | 06:49 | 05:19 | 12:08 |
| | SD | 00:26 | 00:33 | 00:06 | 01:34 | 04:11 | 05:10 |
| 2x | Mean | 00:57 | 00:32 | 01:29 | 00:55 | 00:51 | 01:47 |
| | SD | 00:01 | 00:05 | 00:06 | 00:03 | 00:10 | 00:10 |
| 3x | Mean | 00:42 | 00:27 | 01:09 | 00:43 | 00:51 | 01:34 |
| | SD | 00:11 | 00:06 | 00:10 | 00:08 | 00:03 | 00:10 |

The data show that dilution of the bulk decreased the reconstitution time Similar reconstitution times were obtained when preparing the bulk mixture by mixing the first solution and the second solution at a ratio of 1:1 (v/v) (“2×”) or 2:1 (v/v) (“3×”) (Table 2).

In view of the above data, the lyophilized pharmaceutical formulations prepared with low or medium molecular weight HA and by mixing the first solution and the second solution at a ratio of 1:1 (v/v) (“2×”) were preferred in order to obtain a homogenous formulation for injection in combination with a satisfying reconstitution time.

4.4 Cake Reconstituted with Water

Viscosity of the Formulation

The viscosity was assessed using a microVISC™ viscometer (RheoSense, CA, USA) according to the method of the supplier.

Before each use of the microVISC™, the viscosity of a reference oil was measured to assess the calibration of the equipment.

The sensor cartridge HB02 was first placed into the viscometer. Then, 400 μl of sample were loaded into the disposable pipette which was further mounted on the viscometer.

The advanced parameters were:
Shear Rate=111.6 s−1
Measuring volume=30 μl
Priming volume=15 μl
Pause time=5 s
Range of sensor=60 to 5000 cP

As temperature is a well-known parameter that influences viscosity, each measure has to be performed at 25.0±0.1° C.

The measuring chip contained a rectangular slit flow channel constructed of borosilicate glass, with a uniform cross-sectional area. The sample was injected at a constant flow rate though the flow channel where multiple pressure sensors mounted within the base monitor the pressure drop from the inlet to the outlet. The pressure drop was correlated with the shear-stress at the boundary wall. The shear rate and shear stress were directly related to the geometry of the rectangular slit and the flow rate which allow for viscosity measurements.

A VROC® chip assessed the viscosity by measuring the pressure drop as a test liquid flowed through its rectangular slit microfluidic channel Based on Hagen-Poiseuille flow, it is a well-known application of rheometric principles (K. Walters, Rheometry, Chapman and Hall, London, 1975), that is also listed in US Pharmacopeia.

The viscosity data were exported into the microVISC™ control 2.0 software.

The results of the viscosity measurements are provided in Table 3.

TABLE 3
Viscosity (cP), scale (%), and R² of lyophilized pharmaceutical formulations
according to embodiments of the invention (n = 5); Bulk mixture
of the formulations before lyophilisation: 1x, 2x, or 3x

| | | HA Low MW | | | HA Medium MW | | | HA High MW | | |
|---|---|---|---|---|---|---|---|---|---|---|
| | | Viscosity | Scale | R² | Viscosity | Scale | R² | Viscosity | Scale | R² |
| 1x | Mean | 11.277 | 0.7 | 0.982 | 314.4 | 18.3 | 1.000 | 725.0 | 58.920 | 0.995 |
| | SD | 3.865 | 0.1 | 0.015 | 39.6 | 2.3 | 0.000 | 115.8 | 9.356 | 0.004 |
| 2x | Mean | 8.085 | 0.5 | 0.989 | 278.9 | 16.2 | 1.000 | 863.1 | 75.900 | 0.999 |
| | SD | 0.292 | 0.2 | 0.007 | 7.4 | 0.1 | 0.001 | 58.0 | 7.105 | 0.001 |
| 3x | Mean | 6.944 | 0.4 | 0.992 | 266.1 | 15.7 | 1.000 | 763.2 | 67.020 | 0.993 |
| | SD | 1.332 | 0.1 | 0.009 | 7.9 | 0.5 | 0.000 | 121.8 | 13.283 | 0.007 |

The viscosity increased with increasing HA MW weight: viscosity_{HA high MW}>viscosity_{HA medium MW}>viscosity_{HA low WV}. An optimal viscosity for injection of between 200 cP to 500 cP was found for reconstituted formulations containing medium molecular weight HA.

The viscosity was not influenced by other parameters such as dilution of the bulk mixture.

In conclusion, the lyophilized pharmaceutical formulation containing medium molecular weight HA and prepared by mixing the first solution and the second solution at a ratio of 1:1 (v/v) (“2×”) had a satisfying reconstitution time, while at the same time having a viscosity after reconstitution which both allows easy administration by injection and provides sufficient lubricating action after administration.

Protein Content

The protein content was determined by colorimetric assay using a commercial kit (Detergent Compatible Protein assay kit from Biorad, ref #500-0116) based on manufacturer's recommendation. Briefly, a standard curve with protein standard solution dilutions (Biorad, Quick Start Bovin Serum Albumin Standard, #500-020) was performed. Five μl of standard and reconstituted cake solutions were placed in the well of a clean and dry microplate and 25 μl/well of Reagent A were added. Then, 200 μl of Reagent B were and the microplate were stirred for 5 seconds. After an incubation of 15 minutes, the plates are red at 620 nm.

The results of the protein concentration measurements are provided in Table 4.

TABLE 4
Protein concentration (mg/ml) of lyophilized pharmaceutical formulations
according to embodiments of the invention (n = 5); Bulk mixture
of the formulations before lyophilisation: 1x, 2x, or 3x

| | | HA Low MW | HA Medium MW | HA High MW |
|---|---|---|---|---|
| 1x | Mean | 53.16 | 53.95 | 48.35 |
| | SD | 1.87 | 9.22 | 3.27 |
| 2x | Mean | 50.43 | 45.24 | 40.59 |
| | SD | 1.20 | 2.20 | 3.27 |
| 3x | Mean | 48.99 | 50.08 | 50.70 |
| | SD | 2.07 | 3.53 | 2.38 |

The concentration of the plasmatic proteins was on average 50 mg/ml. Hence, each lyophilized formulation contained about 120 mg of plasmatic proteins (i.e. 50 mg/ml×2.4 ml reconstitution volume). The total weight of the lyophilized formulation was about 200 mg and hence each vial contained about 60% wt of plasmatic proteins.

The variability from sample to sample and between different conditions may be related to the experiment itself (e.g., weights of starting material).

1. A lyophilized pharmaceutical formulation, the formulation comprising:
lyophilized plasma and
lyophilized hyaluronic acid or a derivative thereof,
wherein the hyaluronic acid or derivative thereof has a molecular mass ranging from 0.6 MDa to 1.0 MDa,
wherein the lyophilized formulation has a density between 0.04 g/cm³ and 0.08 g/cm³, and
wherein the formulation, when reconstituted in an aqueous solution, has a reconstitution time of 15 minutes or less and is configured for injection.

2. The lyophilized pharmaceutical formulation of claim 1, wherein the reconstituted pharmaceutical formulation is characterized by a viscosity of between 200 cP and 500 cP.

3. The lyophilized pharmaceutical formulation of claim 1, wherein the lyophilized formulation further comprises an alpha-2 adrenergic receptor agonist.

4. The lyophilized pharmaceutical formulation of claim 1, wherein the formulation comprises from about 30% to about 80% by weight of plasmatic proteins.

5. The lyophilized pharmaceutical formulation of claim 4, wherein the plasmatic proteins are solvent/detergent-treated (S/D) plasma proteins, and/or wherein the derivative of hyaluronic acid is a salt of hyaluronic acid, an ester of hyaluronic acid with an alcohol of the aliphatic, heterocyclic or cycloaliphatic series, or a sulphated form of hyaluronic acid.

6. The lyophilized pharmaceutical formulation of claim 1, further comprising at least one salt; and/or further comprising at least one buffer component or acidic component.

7. The lyophilized pharmaceutical formulation of claim 1, wherein one administration dose comprises:
from 1 mg to 100 mg of the hyaluronic acid or derivative thereof.

8. The lyophilized pharmaceutical formulation of claim 1, wherein the formulation comprises:
from about 30% to about 80% by weight of plasmatic proteins; and
from about 5.0% to about 20.0% by weight of the hyaluronic acid or derivative thereof;
and optionally
from about 0.01% to about 0.1% by weight of an alpha-2-adrenergic receptor agonist;
from about 1.5% to about 3.0% by weight of a salt; and/or
from about 0.1% to about 2.0% by weight of a buffer component or acidic component.

9. (canceled)

10. A process for preparing a lyophilized pharmaceutical formulation, the process comprising:
(a) mixing plasma and hyaluronic acid or derivative thereof, thereby obtaining a bulk mixture, wherein the hyaluronic acid or derivative thereof has a molecular mass ranging from 0.6 MDa to 1.0 MDa;
(b) sterilizing the bulk mixture by steam sterilization or filter sterilization, thereby obtaining a sterile mixture; and
(c) lyophilizing the sterile mixture, thereby obtaining the lyophilized pharmaceutical formulation;
wherein step (a) comprises the steps of
(a1) dissolving the hyaluronic acid or derivative in an aqueous solution, thereby obtaining a first solution;
(a2) preparing a second solution comprising the plasma, and, optionally, an alpha-2 adrenergic receptor agonist, and
(a3) mixing the first and second solution to obtain the bulk mixture, wherein the first solution and the second solution are mixed in a ratio of at least 1:1 (v/v).

11. The process according to claim 10, wherein the bulk mixture has a concentration of plasmatic proteins of 20 mg/ml to 50 mg/ml and a concentration of the hyaluronic acid or derivative thereof of 4 mg/ml to 8 mg/ml.

12. The process according to claim 10, wherein step (a) further comprises mixing an alpha-2 adrenergic receptor agonist, a salt, and/or a buffer component or acidic component, thereby obtaining a bulk mixture having a concentration of the alpha-2 adrenergic receptor agonist of 20 μg/ml to 35 μg/ml, a concentration of the salt of 0.5 mg/ml to 1.5 mg/ml, and/or a concentration of the buffer component or acidic component of 0.05 mg/ml to 3.0 mg/ml.

13. (canceled)

14. A method of administering a composition to a subject, the method comprising: administering to the subject the lyophilized pharmaceutical formulation of claim 1.

15. (canceled)

16. The lyophilized pharmaceutical formulation of claim 2, wherein the reconstituted pharmaceutical formulation is characterized by a viscosity of between 250 cP and 400 cP.

17. The lyophilized pharmaceutical formulation of claim 3, wherein the alpha-2 adrenergic receptor agonist is clonidine or a derivative thereof.

18. The lyophilized pharmaceutical formulation of claim 5, wherein the plasmatic proteins are human S/D plasma proteins.

19. The lyophilized pharmaceutical formulation of claim 6, wherein the salt is a calcium salt, wherein the salt is calcium chloride, and/or wherein the acidic component is hydrochloric acid.

20. The method according to claim 14, wherein the subject suffers from a musculoskeletal disease.

21. The lyophilized pharmaceutical formulation of claim 7, the one administration dose comprises from 5 mg to 40 mg of the hyaluronic acid or derivative thereof.

22. The lyophilized pharmaceutical formulation of claim 7, the one administration dose comprises from 1 μg to 500 μg of an alpha-2-adrenergic receptor agonist.

23. The lyophilized pharmaceutical formulation of claim 7, the one administration dose comprises from 5 μg to 125 μg of an alpha-2-adrenergic receptor agonist.
